SEQUENCE LISTING (34 sequences)

SEQ ID NO: 1 - DNA, 21 bp, Artificial sequence
  Amplification primer
cgatggagtccttgcctacaa

SEQ ID NO: 2 - DNA, 23 bp, Artificial sequence
  Amplification primer
cgcctatgcctgtctaaccatgc

SEQ ID NO: 3 - DNA, 20 bp, Artificial sequence
  Amplification primer
tactcacgcctcgaaaacct

SEQ ID NO: 4 - DNA, 20 bp, Artificial sequence
  Amplification primer
cagcgacttcctcactttcc

SEQ ID NO: 5 - DNA, 20 bp, Artificial sequence
  Amplification primer
ccccctccactccaataaat

SEQ ID NO: 6 - DNA, 20 bp, Artificial sequence
  Amplification primer
ccaggagccactgaagactc

SEQ ID NO: 7 - DNA, 20 bp, Artificial sequence
  Amplification primer
cagtttggagctcctggaag

SEQ ID NO: 8 - DNA, 24 bp, Artificial sequence
  Amplification primer
ttaccttcagctgccacttctacg

SEQ ID NO: 9 - DNA, 20 bp, Artificial sequence
  Amplification primer
ggtggaaaaccaggtaagca

SEQ ID NO: 10 - DNA, 20 bp, Artificial sequence
  Amplification primer
gaggaagaggaggaggagga

SEQ ID NO: 11 - DNA, 20 bp, Artificial sequence
  Amplification primer
ggttcttctgcgctactgct

SEQ ID NO: 12 - DNA, 19 bp, Artificial sequence
  Amplification primer
ccgccggaagcccaactat

SEQ ID NO: 13 - DNA, 20 bp, Artificial sequence
  Amplification primer
cgacccaacccaagaatcta

SEQ ID NO: 14 - DNA, 20 bp, Artificial sequence
  Amplification primer
gaaggcctaagcacaacagc

SEQ ID NO: 15 - DNA, 24 bp, Artificial sequence
  Amplification primer
aataccgagttaggcgcagtatgg

SEQ ID NO: 16 - DNA, 24 bp, Artificial sequence
  Amplification primer
taatctcgagaccaacgtggagga

SEQ ID NO: 17 - DNA, 22 bp, Artificial sequence
  Amplification primer
atgctcctttcctcctgaaacc

SEQ ID NO: 18 - DNA, 21 bp, Artificial sequence
  Amplification primer
ccaccagacgctgtttagtca

SEQ ID NO: 19 - DNA, 22 bp, Artificial sequence
  Amplification primer
taaatccatgctgagaattgca

SEQ ID NO: 20 - DNA, 20 bp, Artificial sequence
  Amplification primer
gtctgctttcctccctgatg

SEQ ID NO: 21 - DNA, 20 bp, Artificial sequence
  Amplification primer
ggagcggttagggctactct

SEQ ID NO: 22 - DNA, 20 bp, Artificial sequence
  Amplification primer
aaaattctcccatgggcttc

SEQ ID NO: 23 - DNA, 20 bp, Artificial sequence
  Amplification primer
cctttcccaggttctcttcc

SEQ ID NO: 24 - DNA, 20 bp, Artificial sequence
  Amplification primer
tgcaaatccagggtgcagtg

SEQ ID NO: 25 - DNA, 24 bp, Artificial sequence
  Amplification primer
gccttggcaatcatcttgctcttc

SEQ ID NO: 26 - DNA, 20 bp, Artificial sequence
  Amplification primer
ccttctcctctgccatcttg

SEQ ID NO: 27 - DNA, 20 bp, Artificial sequence
  Amplification primer
gagatggaagggggaaagag

SEQ ID NO: 28 - DNA, 20 bp, Artificial sequence
  Amplification primer
tagggctgctggaaggtaaa

SEQ ID NO: 29 - DNA, 20 bp, Artificial sequence
  Amplification primer
ccaggagagagtcccagatg

SEQ ID NO: 30 - DNA, 20 bp, Artificial sequence
  Amplification primer
aggctgtgccttcctacaga

SEQ ID NO: 31 - DNA, 20 bp, Artificial sequence
  Amplification primer
acggtgtacacagcattcca

SEQ ID NO: 32 - DNA, 24 bp, Artificial sequence
  Amplification primer
aataccgagttaggcgcagtatgg

SEQ ID NO: 33 - DNA, 24 bp, Artificial sequence
  Amplification primer
tggtcaggacattagcgccttctt

SEQ ID NO: 34 - DNA, 22 bp, Artificial sequence
  Amplification primer
tgaactgggcagctatgaagtc

(19) United States
(12) Patent Application Publication
(10) Pub. No.: US 2008/0095761 A1
(43) Pub. Date: Apr. 24, 2008

(54) Hedgehog Signaling in Prostate Regeneration Neoplasia and Metastasis

(75) Inventors: Philip A. Beachy, Towson, MD (US); David M. Berman, Towson, MD (US); Sunil S. Karhadkar, Towson, MD (US)

Correspondence Address: DLA PIPER US LLP, 4365 EXECUTIVE DRIVE, SUITE 1100, SAN DIEGO, CA, 92121-2133, US

(73) Assignee: THE JOHNS HOPKINS UNIVERSITY, Baltimore, MD (US)

(21) Appl. No.: 10/572,430

(22) PCT Filed: Oct. 1, 2004
(86) PCT No.: PCT/US04/32087

§ 371 Date: Feb. 2, 2007

Related U.S. Application Data
(60) Provisional application No. 60/507,588, filed on Oct. 1, 2003.
(60) Provisional application No. 60/552,542, filed on Mar. 12, 2004.

(51) Int. Cl. A61K 39/395; A61K 31/4355; A61K 31/7105; C12Q 1/48; C12Q 1/68; C12Q 1/02; A61K 38/16

(52) U.S. Cl. 424/130.1; 435/15; 435/29; 435/6; 435/7.1; 514/12; 514/278; 514/44

(57) ABSTRACT

Elevated Hedgehog (Hh) pathway activity, including ligand stimulated Hh pathway activity, was detected in prostate tumors, and determined to be associated with growth and proliferation of the cancer cells. Accordingly, methods are provided for treating a prostate cancer associated with elevated Hh pathway activity by reducing or inhibiting the Hh pathway activity. Also provided are methods of determining the responsiveness of a prostate tumor to treatment with an Hh pathway antagonist.

### CROSS REFERENCE TO RELATED APPLICATIONS

This application claims the benefit of priority under 35 U.S.C. § 119(e) of U.S. Ser. No. 60/507,588, filed Oct. 1, 2003, and U.S. Ser. No. 60/552,542, filed Mar. 12, 2004, the entire contents of which are incorporated herein by reference.

### BACKGROUND OF THE INVENTION

1. Field of the Invention

The present invention relates generally to the use of compounds to treat a variety of disorders, diseases and pathologic conditions and more specifically to the use of Hedgehog antagonists for inhibiting hedgehog pathway activity in prostate cancer.

2. Background Information

Pattern formation is the activity by which embryonic cells form ordered spatial arrangements of differentiated tissues. Speculation on the mechanisms underlying these patterning effects usually centers on the secretion of a signaling molecule that elicits an appropriate response from the tissues being patterned. More recent work aimed at the identification of such signaling molecules implicates secreted proteins encoded by individual members of a small number of gene families.

Members of the Hedgehog family of signaling molecules mediate many important short- and long-range patterning processes during invertebrate and vertebrate development. Exemplary hedgehog genes and proteins are described in PCT publications WO 95/18856 and WO 96/17924. The vertebrate family of hedgehog genes includes at least four members, three of which, herein referred to as Desert hedgehog (Dhh), Sonic hedgehog (Shh) and Indian hedgehog (nhh), apparently exist in all vertebrates, including fish, birds, and mammals. A fourth member, herein referred to as tiggie-winkle hedgehog (Tbh), appears specific to fish. Desert hedgehog (Dhh) is expressed principally in the testes, both in mouse embryonic development and in the adult rodent and human; Indian hedgehog (Imh) is involved in bone development during embryogenesis and in bone formation in the adult; and, Shh is primarily involved in morphogenic and neuroinductive activities. Given the critical inductive roles of hedgehog polypeptides in the development and maintenance of vertebrate organs, the identification of hedgehog interacting proteins and their role in the regulation of gene families known to be involved in cell signaling and intercellular communication provides a possible mechanism of tumor suppression.

Prostatic adenocarcinoma is the most commonly diagnosed non-cutaneous cancer for men in the United States. The incidence is likely to continue to increase as people survive longer and more middle-aged men undergo routing screening for the disease. Men diagnosed with early stage small volume disease have the best outcome following curative treatment. Therefore the aim of early detection programs is to diagnose cancer at an early curable stage.

The role of Hh pathway activity in promoting metastatic growth suggests that pathway antagonists may offer significant therapeutic improvements in the treatment of advanced prostate cancer. The ability to modulate one or more genes that are part of the hedgehog signaling cascade thus represents a possible therapeutic approach to several clinically significant cancers. A need therefore exists for methods and compounds that inhibit signal transduction activity by modulating activation of a hedgehog, patched, or smoothlened-mediated signal transduction pathway, such as the Hedgehog signaling pathway, to reverse or control aberrant growth related to prostate cancer.

### SUMMARY OF THE INVENTION

The present invention is based, in part, on the determination that Hedgehog (Hh) pathway activity is elevated in prostate tumor cells as compared to corresponding normal cells of the organ with the tumor, and that agents that decrease the Hh pathway activity inhibit proliferation or metastasis of prostate tumor cells. Hh ligands that can stimulate Hh pathway activity include Sonic hedgehog (SHH), Indian hedgehog (IHH), and/or Desert hedgehog (DHH). Elevated Bh pathway activity also can be due, for example, to a mutation in a Hh ligand receptor such as Patched (PTCH), wherein PTCH in inactivated, resulting in unregulated Smoothened (SMO) activity and elevated Hh pathway activity. Accordingly, the present invention provides methods of treating a prostate tumor characterized by elevated Hh pathway activity, as well as methods of determining whether a prostate tumor has such activity and methods of identifying agents useful for treating such tumors. As such, methods of personalized medicine are provided, wherein agents can be selected that are particularly useful for treating a particular prostate tumor in a patient.

The present invention relates to a method of reducing or inhibiting proliferation or metastasis of cells of a prostate tumor characterized by elevated Hh pathway activity. Such a method can be performed, for example, by contacting the cells with at least one (e.g., 1, 2, 3, 4, or more) Hh pathway antagonist, whereby proliferation or metastasis of the cells of the prostate tumor is reduced or inhibited. The Hh pathway generally includes an Hh ligand (e.g., SHH, IHH and/or DHH), which binds an Hh ligand receptor (e.g., PTCH), resulting in activation of SMO (a G protein coupled receptor-like polypeptide), which transduces the Hh signal downstream, resulting in activation of additional members of the Hh pathway (e.g., Fused), including Hh pathway stimulated transcription factors (e.g., members of the GLI family of transcription factors). Also associated with Hh pathway activity are transcriptional targets, including, for example, nestin and BMI-1, which can be induced by activated GLI transcription factor. As such, it will be recognized that a Hh pathway antagonist useful in a method of the invention is selected, in part, in that it acts at or downstream of the position in the Hh pathway associated with the elevated Hh pathway activity. For example, where elevated Hh pathway activity is ligand stimulated, the Hh antagonist can be selected based on the ability, for example, to sequester the Hh ligand or to reduce or inhibit binding of the Hh ligand to its receptor, or at any point downstream of these events. In comparison, where elevated Hh pathway activity is due to an inactivating mutation of the Hh ligand receptor (e.g., PTCH), the Bh pathway antagonist can be selected based on the ability, for example, to bind to and inhibit SMO or to reduce the activity of an activating GLI transcription factor (e.g., GLI-1 or GLI-2), but not at a point upstream.

Thus, in one embodiment, the invention provides a method of ameliorating a prostate tumor in a subject. Such a method can be performed by administering to the subject at least one Hh pathway antagonist such that the Hh pathway antagonist contacts cells of the tumor in the subject. According to the present method, the Hh pathway antagonist(s) can reduce or inhibit proliferation or metastasis of the tumor cells, thereby ameliorating the prostate tumor in the subject.

A prostate tumor in a subject to be treated can be any prostate tumor that exhibits elevated Hh pathway activity (e.g., elevated ligand stimulated Hh pathway activity). In one embodiment, the tumor is a malignant tumor. Hh pathway antagonist(s) can be administered in any way typical of an agent used to treat the particular type of prostate tumor. For example, the Hh pathway antagonist(s) can be administered orally or parenterally, including, for example, by injection or as a suppository, or by any combination of such methods.

The Hh pathway antagonist can be any type of compound as disclosed herein or otherwise having the ability to interfere with Hh pathway activity. In one embodiment, the Hh pathway antagonist is an antibody, for example, an antibody specific for one or more Hh ligand(s) (e.g., an anti-SHH, anti-IHH, and/or anti-DHH antibody). In another emdociment, the Hh pathway antagonist is a SMO antagonist such as a steroidal alkaloid, or a derivative thereof (e.g., cyclopamine or jervine), or other synthetic small molecule such as SANT-1, SANT-2, SANT-3, or SANT-4. In still another embodiment, a combination of Hh pathway antagonists are administered to the subject. Further, any additional compounds that can provide a therapeutic benefit can be administered to the subject, including, for example, a chemotherapeutic agent or nutritional supplement, and/or the subject can be further treated, for example, by radiation therapy or using a surgical procedure.

The present invention further relates to a method of identifying a prostate tumor of a subject amenable to treatment with a Hh pathway antagonist. As such, the method provides a means to determine whether a subject having a prostate tumor is likely to be responsive to treatment with an Hh pathway antagonist. The method can be performed, for example, by detecting elevated Hh pathway activity in a sample of cells of the prostate tumor of the subject as compared to corresponding normal cells, wherein detection of an elevated level indicates that the subject can benefit from treatment with an Hh pathway antagonist. The sample of cells can be any sample, including, for example, a tumor sample obtained by biopsy of a subject having the tumor, a tumor sample obtained by surgery (e.g., a surgical procedure to remove and/or debulk the tumor), or a sample of the subject's bodily fluid. The Hh pathway activity can be elevated due, for example, to a mutation of a gene encoding an Hh pathway polypeptide (e.g., an inactivating mutation of PTCH), or can be elevated ligand stimulated Hh pathway activity.

In one embodiment, the method of identifying a prostate tumor amenable to treatment with a Hh pathway antagonist includes detecting an abnormal level of expression of one or more Hh pathway polypeptide(s), including, for example, one or more Hh ligands (e.g., SHH, IHH, and/or desert hedgehog), Hh ligand receptors (e.g., PTCH), or transcription factors (a GLI family member). In one embodiment, the abnormal expression is an elevated expression of one or more Hh pathway polypeptide(s), including, for example, one or more Hh ligands (e.g., SHH, IHH, and/or desert hedgehog), Hh ligand receptors (e.g., PTCH), or transcription factors (a GLI family member), or a combination of such Hh pathway polypeptides. In another embodiment, the abnormal level of expression is a lower expression of one or more Hh pathway polypeptide(s), including, for example, GLI-3, which acts as a transcriptional repressor in the Hh pathway. Increased or decreased expression of an Hh pathway polypeptide can be detected by measuring the level of a polynucleotide encoding the Hh pathway polypeptide using, for example, a hybridization assay, a primer extension assay, or a polymerase chain reaction assay (e.g., measuring the level of PTCH mRNA expression and/or GLI mRNA expression); or by measuring the level the Hh pathway polypeptide(s) using, for example, an immunoassay or receptor binding assay.

In another embodiment, the method of identifying a prostate tumor amenable to treatment with a Hh pathway antagonist includes detecting an elevated activity of one or more Hh pathway polypeptide(s). For example, elevated activity of Hh pathway transcription factor (e.g., a GLI family member) can be detected by measuring increased binding activity of the transcription factor to a cognate transcription factor regulatory element (e.g., using an electrophoretic mobility shift assay); by measuring increased expression of a reporter gene comprising a cognate transcription factor regulatory element; or measuring expression of GLI and/or of PTCH, and/or a target of the GLI transcription factor (e.g., by detecting transcription of nestin or BMI-1). In still another embodiment, the method can include detecting expression of an Hh pathway polypeptide having an inactivating mutation, wherein the mutation is associated with elevated Hh pathway activity (e.g., by detecting expression of a mutant PTCH Hh ligand receptor).

The method of identifying a prostate tumor amenable to treatment with a Hh pathway antagonist can futher include contacting cells of the sample with at least one Hh pathway antagonist, and detecting a decrease in Hh pathway activity in the cells following said contact. The decreased Hh pathway activity can be detected, for example, by measuring decreased expression of a reporter gene regulated by an Hh pathway tanscription factor, or by detecting a decrease in proliferation of the tumor cells. Such a method provides a means to confimi that the prostate tumor is amenable to treatment with an Hh pathway antagonist. Further, the method can include testing one or more different Hh pathway antagonists, either alone or in combination, thus providing a means to identify one or more Hh pathway antagonists useful for treating the particular prostate tumor being examined.

The present invention further relates to a method of identifying an agent useful for treating a prostate tumor having elevated Hh pathway activity. In one embodiment, the method provides a means for practicing personalized medicine, wherein treatment is tailored to the particular patient based on the characteristics of the prostate tumor in the patient. The present method can be practiced, for example, by contacting a sample of cells of a prostate tumor with at least one test agent, wherein a decrease in Hh pathway activity in the presence of the test agent as compared to Hh pathway activity in the absence of the test agent identifies the agent as useful for treating the prostate tumor.

The present method can be practiced using test agents that are known to be effective in treating a prostate tumor having elevated Hh pathway activity in order to identify one or more agents that are particularly useful for treating the prostate tumor being examined, or using test agents that are being examined for effectiveness. As such, in one aspect, the test agent examined according to the present method can be any type of compound, including, for example, a peptide, a polynucleotide, a peptidomimetic, or a small organic molecule, and can be one of a plurality of similar but different agents (e.g., a combinatorial library of test agents, which can be a randomized or biased library or can be a variegated library based on known effective agent). In another aspect, the test agent comprises a known Hh pathway antagonist such as an antibody (e.g., an anti-SHH antibody and/or anti-IHH antibody), a steroidal alkaloid or a derivative thereof (e.g., cyclopamine, jervine, or triparanol), or a combination thereof.

Generally, though not necessarily, the method is performed by contacting the sample of cells ex vivo, for example, in a culture medium or on a solid support. As such, the methods are conveniently adaptable to a high throughput format, wherein a plurality (i.e., 2 or more) of samples of cells, which can be the same or different, are examined in parallel. Thus in one embodiment, test agents can be tested on several samples of cells from a single patient, allowing, for example, for the identification of a particularly effective concentration of an agent to be administered to the subject, or for the identification of a particularly effective agent to be administered to the subject. In another embodiment, a high throughput format allows for the examination of two, three, four, etc., different test agents, alone or in combination, on the cells of a subject's prostate tumor such that the best (most effective) agent or combination of agents can be used for a therapeutic procedure. Accordingly, in various embodiments, the high throughput method is practiced by contacting different samples of cells of different subjects with same amounts of a test agent; or contacting different samples of cells of a single subject with different amounts of a test agent; or contacting different samples of cells of two or more different subjects with same or different amounts of different test agents. Further, a high throughput format allows, for example, control samples (positive controls and or negative controls) to be run in parallel with test samples, including, for example, samples of cells known to be effectively treated with an agent being tested. Variations of the exemplified methods also are contemplated.

### BRIEF DESCRIPTION OF THE DRAWINGS

FIG. 1 shows autonomous Hh stimulation in growth of human prostate cancer cell lines.

FIG. 1a is a pictoral diagram indicating expression of Indian (IHH) and Sonic (SHH) ligands in benign prostate epithelial (PrE) cells and in all prostate cancer cell lines examined (CWR-22RV1 is abbreviated as 22RV1) and a graphical representation showing transcripts encoding the Hh pathway targets PTCH and GLI present in cancer cell lines.

FIG. 1b is a graphical representation showing quantitative RT-PCR for PTCH performed on RNA from these samples and normalized to phosphoglycerate kinase (PGK).

FIG. 1c is a graphical representation showing normalized expression of a Hh-responsive reporter in human prostate cancer cells and modulation by cyclopamine, Sonic hedgehog ligand (ShhNp), and 5E1 neutralizing antibody.

FIG. 1d is a graphical representation showing dose-dependent inhibition of growth in prostate cancer cells.

FIG. 1e is a graphical representation showing inhibition of PC3 cell growth when cultured in increasing concentrations of 5E1 and opposite effects of Hh ligand stimulation.

FIG. 1f is a graphical representation showing decreased expression of transcripts encoding the cell proliferation regulators c-myc.

FIG. 1g is a graphical representation showing decreased expression of transcripts encoding the cell proliferation regulators cyclin D1.

FIG. 1h is a graphical representation showing decreased expression of transcripts encoding the cell proliferation regulators as well as the progenitor cell marker nestin upon Hh pathway blockade.

FIG. 2 shows complete and durable regression of metastasis-derived human prostate tumors upon Hh pathway blockade.

FIG. 2a is a graphical representation showing xenograft tumors from PC3, 22RV1, and 22RV1-GLI grown to a median size of 155 mm³ prior to treatment.

FIG. 2b is a graphical and pictoral respresentation showing antibodies against the Ki-67 proliferation antigen resulting in a 90% reduction in proliferation index in PC3 xenografts treated for nine days with 10 mg/kg cyclopamine as compared to vehicle-treated tumors.

FIG. 2c is a graphical and pictoral representation showing durable regression of PC3 (c) and 22RV1.

FIG. 2d is a graphical representation showing prostate cancer xenografts after 28 days(PC3) and 22 days(22RV1) of high dose (50 mg/kg) cyclopamine treatment.

FIG. 3 shows that Hh pathway activity is required for regeneration of prostate epithelium.

FIG. 3a is a graphical representation showing the experimental timeline.

FIG. 3b is a graphical representation showing that the wet weights of prostate glands decreased 3-fold in vehicle-treated male castrates, and that Hh pathway blockade with cyclopamine (50 mg/kg/day, subcutaneous injection) completely blocked prostate regeneration.

FIG. 3c is a pictoral representation showing large, convoluted prostate glands with tall columnar epithelium in intact animals and in DHT-treated castrates, whereas glands from vehicle-treated castrates and from castrates treated with DHT and cyclopamine are significantly smaller and simpler and have lower (cuboidal) epithelium. Scale bar=200 μM.

FIG. 4 shows elevated Hh pathway activity in human prostate cancer metastasis.

FIG. 4a is a pictoral representation indicating universal expression of Indian (IHH) and Sonic (SHH) ligands in benign tissue from surgically resected prostates (n=12), in adjacent locally growing prostate cancer (n=12), and in prostate cancer metastasis removed at autopsy (n=16 samples from 13 patients).

FIG. 4b shows graphical representations of quantitative RT-PCR for PTCH performed on RNA from these samples indicating a high level of Hh pathway activity in metastasis and much lower (>10-fold less) Hh pathway activity in 25% of localized tumors (note change of scale in y-axis). Levels are normalized to PGK and expressed as fold-elevation of PTCH relative to benign epithelial cells.

FIG. 5 shows that Hh pathway activity determines metastatic potential in Dunning rat prostate carcinoma cell variants.

FIG. 5a is a graphical representation showing a high level Hi-responsive Gli-luciferase reporter activity in the highly metastatic lines (Mat-LyLu, AT3.1, and AT6.3), whereas lines with low metastatic potential (G, AT1, and AT2) expressed only modest levels of reporter activity.

FIG. 5b is a graphical representation showing a higher baseline Hh reporter activity and greater responsiveness to added ligand (ShhNp) in highly metastatic AT6.3 cells as compared to low-level reporter activity and attenuated ligand response in poorly metastatic AT2.1 cells.

FIG. 5c is a graphical representation showing complete growth inhibition and reduced viability of AT6.3 cells treated with cydopamine as compared to milder growth effects in AT2.1 cells.

FIG. 5d is a pictoral representation showing widespread metastasis after subcutaneous inoculation of AT6.3 cells in vehicle-treated control mice after 10-days (viscera and lungs). Arrows indicate some of the metastasis.

FIG. 5e is a pictoral representation showing an AT6.3 inoculated animal after 30 days of cydopamine treatment.

FIG. 5f is a pictoral representation showing non-metastatic AT2.1 cells becoming rapidly metastatic (lungs are shown 13 days after inoculation) upon stable overexpression of GLI.

FIG. 5g is a graphical representation showing survival of nude mice bearing subcutaneous Dunning prostate carcinoma xenografts.

FIG. 6 shows that Hh, pathway activation drives a metastasis-promoting program of cell invasiveness and gene expression.

FIG. 6a is a pictoral representation showing numerous AT2.1-GLI cells that have invaded a Matrigel-coated membrane after 21 hours. Scale bar=100 μM.

FIG. 6b is a graphical representation showing that poorly metastatic AT2.1 cells rarely invaded the membrane, whereas highly metastatic AT2.1 GLI cells and AT6.3 cells invaded readily. Invasion was suppressed in AT6.3 cells by cyclopamine blockade of Hh pathway activity.

FIG. 6c is a graphical representation showing that invasiveness was also blocked in human 22RV1 prostate cancer cells by Hh pathway blockade, either with cyclopamine or with 5E1 neutralizing antibody. Invasiveness of AT2.1-GLI and 22RV1-GLI cells was not affected by cyclopamine.

FIG. 6d is a graphical representation showing quantitative RT-PCR for transcripts encoding the metastasis-associated mesenchymal transcriptional repressor Snail. Hh pathway blockade with cyclopamine lead to decreased expression of Snail.

FIG. 6e is a graphical representation showing quantitative RT-PCR for transcripts encoding the epithelial adhesion factor E-cadherin. Hh pathway blockade with cyclopamine lead to increased expression of its target, E-cadherin in rat and human metastasis-derived prostate cancer cell lines. Overexpression of GLI resulted in increased Snail and decreased E-cadherin expression in AT-2.1-GLI cells.

FIG. 6f is a graphical representation showing increased expression of the metastasis suppressor Ndrgl in cyclopamine-treated human prostate cancer cells.

### DETAILED DESCRIPTION OF THE INVENTION

The present invention is based on the identification of elevated hedgehog (Hh) pathway activity in tumors derived from the hindgut, a tissue with prominent and diverse roles for Hh signaling in developmental patterning and tissue homeostasis (see Berman et al., Nature 425:846-851, 2003, which is incorporated herein by reference; see, also, (Lanm, M. L. et al. Sonic hedgehog activates mesenchymal Glil expression during prostate ductal bud formation. Dev. Biol. 249, 349-66 (2002); Litingtung, Y., Lei, L., Westphal, H. & Chiang, C. Sonic hedgehog is essential to foregut development [see comments]. Nat. Genet. 20, 58-61 (1998); Berman, D. M. et al. Roles for Hedgehog signaling in androgen production and prostate ductal morphogenesis. Dev. Biol Online (2004); and Freestone, S. H. et al. Sonic hedgehog regulates prostatic growth and epithelial-differentiation. Dev Biol. 264, 352-62 (2003)). Activation of the Hh signaling pathway by sporadic mutations or in familial conditions such as Gorlin syndrome has been associated with tumorigenesis in skin, cerebellum, and skeletal muscle (see Bale, A. E. & Yu, K. P., The hedgehog pathway and basal cell carcinomas. Hum. Mol. Genet. 10, 757-62. (2001); Taipale, J. & Beachy, P. A. The Hedgehog and Wnt signalling pathways in cancer. Nature 411, 349-54. (2001); Wechsler-Reya, R. & Scott, M. P. The developmental biology of brain tumors. Annu. Rev. Neurosci. 24, 385-428 (2001); and Freestone, S. H. et al. Sonic hedgehog regulates prostatic growth and epithelial differentiation. Dev Biol. 264, 352-62 (2003)).

As disclosed herein, Hedgehog (Hh) pathway activity dramatically increases invasiveness of prostate cancer cells and promotes changes in expression of genes known to modulate metastasis. Prostate cancer cells displayed elevated levels of Hh pathway activity that were suppressed by the Hh pathway antagonist cyclopamine. Cyclopamine also suppressed cell growth in vitro and caused regression of xenograft tumors in vivo. Unlike Gorlin syndrome tumors, Hh pathway activity and cell growth in prostate tumors is driven by endogenous expression of Hh ligands, as indicated by the presence of Sonic hedgehog (SHH) and Indian hedgehog (IHH) transcripts, by the pathway-inhibitory and growth-inhibitory activity of an Hh-neutralizing antibody, and by the dramatic growth-stimulatory activity of exogenously added Hh ligand. These results demonstrate that the second most lethal malignancy in men is characterized by elevated Hh pathway activity that is essential for tumor growth. Accordingly, the present invention provides methods of treating a prostate tumor characterized by elevated Hh pathway activity as compared with a normal cell, as well as methods of determining whether a prostate tumor is amenable to treatment using an Hh pathway antagonist, and methods of identifying agents useful for treating such tumors.

The term “agonist” refers to an agent or analog that binds productively to a receptor and mimics its biological activity. The term “antagonist” refers to an agent that binds to receptors but does not provoke the normal biological response. Thus, an antagonist potentiates or recapitulates, for example, the bioactivity of patched, such as to repress transcription of target genes. The term “hedgehog antagonist” as used herein refers not only to any agent that may act by directly inhibiting the normal function of the hedgehog protein, but also to any agent that inhibits the hedgehog signaling pathway, and thus recapitulates the function of ptc. The term “hedgehog agonist” likewise refers to an agent which antagonizes or blocks the bioactivity of patched, such as to increase transcription of target genes.

As used herein, reference to the “Hh pathway” means the Hedgehog signal transduction pathway. The HI pathway is well known (see, e.g., U.S. Pat. No. 6,277,566 B1; U.S. Pat. No. 6,432,970 B2; Lum and Beachy, Science 304:1755-1759, 2004; and Bale and Yu, Hum. Mol. Genet. 10:757-762, 2001, each of which is incorporated herein by reference). Briefly, SHH, IHH and DHH are a family of secreted proteins that act as ligand (Hh ligands) to initiate the Hh pathway, which is involved in morphogenetic development and proliferation of cells in a variety of tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof.

Hh ligands bind to a receptor complex that includes Patched (PTCH; e.g., PTCH-l in humans) and Smoothened (SMO), which are G-protein coupled receptor-like polypeptides. PTCH is an integral membrane protein with twelve transmembrane domains that acts as an inhibitor of SMO activation. Hh ligand binding to PTCH results in activation of SMO (see, e.g., Taipale et al., Nature 418:892-897, 2002, which is incorporated herein by reference), resulting in transduction of the signal and activation of the GLI family of transcriptional activators (e.g., GLI-1 and GLI-2, which act as transcriptional activators, and GLI-3, which acts as a transcriptional repressor), which are homologs of the Drosophila cubitis iiiterruptis gene. Several kinases also are believed to be involved in the Hh pathway between SMO and the GLI transcription factors, including, for example, protein kinase A, which can inhibit GLI activity. Suppressor of Fused (SUFU) also interacts directly with GLI transcription factors to repress their activity. In addition, various transcriptional targets such as nestin and BMI-1 are regulated by Hh pathway activity.

The Hh signaling pathway specifies patterns of cell growth and differentiation in a wide variety of embryonic tissues. Mutational activation of the Hh pathway, whether sporadic or in Gorlin Syndrome, is associated with tumorigenesis in a limited subset of these tissues, predominantly slin, cerebellum, and skeletal muscle (Wechsler-Reya and Scott, The developmental biology of brain tumors. Ann. Rev. Neurosci. 24, 385-428 (2001); Bale and Yu, The hedgehog pathway and basal cell carcinomas. Hum. Mol. Genet. 10, 757-62 (2001)). Known pathway-activating mutations include those that impair the ability of PTCH (the target of Gorlin Syndrome mutations), a transporter-like Hh receptor (Taipale et al., Patched acts catalytically to suppress the activity of Smoothened. Nature 418, 892-7 (2002), to restrain Smoothened (SMO) activation of transcriptional targets via the GLI family of latent transcription factors. Binding of Hh ligand to PTCH is functionally equivalent to genetic loss of PTCH, in that pathway activation by either requires activity of SMO, a seven transmembrane protein that binds to and is inactivated by the pathway antagonist, cyclopamine (Chen et al., Ihibition of Hedgehog signaling by direct binding of cyclopamine to Smoothened. Genes Dev 16, 2743-8 (2002)).

The term “Hh pathway activity” is used herein to refer to the level of Hedgehog pathway signal transduction that is occurring in cells. Hh pathway activity can be determined using methods as disclosed herein or otherwise known in the art (see, e.g., Berman et al., Medulloblastoma growth inhibition by hedgehog pathway blockade. Science 297, 1559-61 (2002); Chen et al., Small molecule modulation of Smoothened activity. Proc Natl Acad Sci USA99, 14071-6 (2002)). As used herein, the term “elevated” or “abnormally elevated”, when used in reference to Hh pathway activity, means that the Hh pathway activity is increased above the level typically found in normal (i.e., not cancer) differentiated cells of the same type as the cells from which the tumor are derived. As such, the term “elevated Hh pathway activity” refers to the level of Hh pathway activity in prostate tumor cells as compared to corresponding normal cells. Generally, elevated Hh pathway activity is at least about 20% (e.g., 30%, 40%, 50%, 60%, 70%, or more) greater than the Hh pathway activity in corresponding normal cells. In this respect, it should be recognized that Hh pathway activity is determined with respect to a population of cells, which can be a population of tumor cells or a population of normal cells, and, therefore, is an average activity determined from the sampled population.

Reference herein to “corresponding normal cells” means cells that are from the same organ and of the same type as the prostate tumor cell type. In one aspect, the corresponding normal cells comprise a sample of cells obtained from a healthy individual. Such corresponding normal cells can, but need not be, from an individual that is age-matched and/or of the same sex as individual providing the prostate tumor cells being examined. In another aspect, the corresponding normal cells comprise a sample of cells obtained from an otherwise healthy portion of tissue of a subject having a prostate tumor.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

Accordingly, the invention provides methods of reducing or inhibiting Hh pathway activity and/or proliferation or metastasis of cells of a prostate tumor characterized by elevated or abnormally elevated Hh pathway activity. As used herein, the terms “reduce” and “inhibit” are used together because it is recognized that, in some cases, a decrease, for example, in Hh pathway activity can be reduced below the level of detection of a particular assay. As such, it may not always be clear whether the activity is “reduced” below a level of detection of an assay, or is completely “inhibited”. Nevertheless, it will be clearly determinable, following a treatment according to the present methods, that the level of Hh pathway activity (and/or cell proliferation or metastasis) is at least reduced from the level before treatment. Generally, contact of prostate tumor cells having elevated Hh pathway activity with an Hh pathway antagonist reduces the Hh pathway activity by at least about 20% (e.g., 30%, 40%, 50%, 60%, 70%, or more). For example, the Hh pathway activity in a prostate tumor cell treated according to the present methods can be reduced to the level of Hh pathway activity typical of a corresponding normal cell.

A Hh pathway antagonist useful in a method of the invention generally acts at or downstream of the position in the Hh pathway that is associated with the elevated Hh pathway activity. For example, where elevated Hh pathway activity is ligand stimulated, the Hh antagonist can be selected based on the ability, for example, to sequester the Hh ligand (e.g., an antibody specific for the Hh ligand) or to reduce or inhibit binding of the Hh ligand to its receptor. Since Hhhligand activity is dependent on autoprocessing of the Hh ligand (e.g., SHH) into a C-terminal fragment, and an N-terminal fragment that is further modified by attachment of cholesterol and palmitate molecules (and constitutes the ligand; see, e.g., Mann and Beachy, Ann. Rev. Biochein. 73:891-923, 2004, which is incorporated herein by reference), ligand stimulated Hh pathway activity also can be reduced or inhibited by inhibiting autocleavage of the Im ligand. Where elevated Hh pathway activity is due to an inactivating mutation of the Hh ligand receptor (e.g., PTCH), the Hh pathway antagonist can be selected based on the ability, for example, to sequester SMO (e.g., an antibody specific for SMO) or to reduce activity of a GLI transcription factor (e.g., a polynucleotide comprising a GLI regulatory element, which can act to sequester GLI); an anti-Hh ligand antibody may not necessarily reduce or inhibit elevated Hh pathway activity due to a mutation of PTCH because Hh ligand acts upstream of the defect in the Hh pathway. Further, steroidal alkaloids, such as cyclopamine, and derivatives thereof, and other small molecules such as SANT-1, SANT-2, SANT-3, and SANT-4 can reduce or inhibit elevated Hh pathway activity by directly repressing SMO activity. In addition, cholesterol can be required for Hh pathway activity and, therefore, agents that reduce the availability of cholesterol, for example, by removing it from cell membranes, can act as Hh pathway antagonists (see, e.g., Cooper et al., Nat. Genet 33:508-513 (2003), which is incorporated herein by reference; see, also, Cooper et al., Nat. Genet. 34:113 (2003)).

A Hh pathway antagonist useful in a method of the invention can be any antagonist that interferes with Hh pathway activity, thereby decreasing the elevated or abnormally elevated Hh pathway in the prostate tumor cells. As such, the Hh pathway antagonist can be a peptide, a polynucleotide, a peptidomimetic, a small organic molecule, or any other molecule. Hh pathway antagonists are exemplified by antibodies, including anti-SHH antibodies, anti-IHH antibodies, and/or anti-DHH antibodies, each of which can bind to one or more Hh ligands and decrease ligand stimulated Bh pathway activity. Hh pathway antagonists are further exemplified by SMO antagonists such as steroidal alkaloids and derivatives thereof, including, for example, cyclopamine and jervine (see, e.g., Chen et al., Genes Devel. 16:2743-2748, 2002; and U.S. Pat. No. 6,432,970 B2, each of which is incorporated herein by reference), and SANT-1, SANT-2, SANT-3, and SANT-4 (see Chen et al., Proc. Natl. Acad. Sci., USA 99:14071-14076, 2002, which is incorporated herein by reference); triparanol provides another example of an agent that can act as an Hh pathway antagonist (see, e.g., U.S. Pat. No. 6,432,970 B2). As exemplified herein, an anti-SHH antibody and cyclopamine effectively reduced elevated Hh pathway activity in prostate tumor cells and reduced viability of the cells in vitro, and cyclopamine suppressed growth of prostate tumor xenografts in nude mice.

In one aspect, the present invention provides a method of ameliorating a prostate tumor comprising cells characterized by elevated or abnormally elevated Hh pathway activity in a subject. As used herein, the term “ameliorate” means that the clinical signs and/or the symptoms associated with the prostate tumor are lessened. The signs or symptoms to be monitored will be characteristic of a particular prostate tumor and will be well known to the skilled clinician, as will the methods for monitoring the signs and conditions. For example, the skilled clinician will know that the size or rate of growth of a tumor can monitored using a diagnostic imaging method typically used for the particular prostate tumor (e.g., using ultrasound or magnetic resonance image (MRI) to monitor a prostate tumor).

A prostate tumor for which Hh pathway activity and cell proliferation or metastasis can be reduced or inhibited can be any tumor of the prostate that is characterized, at least in part, by Hh pathway activity that is elevated above levels that are typically found in a normal cell corresponding to the tumor cell. As such, the prostate tumor, which can be a benign tumor or can be a malignant tumor, is exemplified herein by prostate carcinoma, prostatic intraepithelial neoplasia, leiomyosarcoma, and rhabdomyosarcoma, each of which is characterized, in part, by elevated or abnormally elevated ligand stimulated Hh pathway activity and increased expression of the HI ligands SHH and/or IHH.

An agent useful in a method of the invention can be any type of molecule, for example, a polynucleotide, a peptide, a peptidomimetic, peptoids such as vinylogous peptoids, a small organic molecule, or the like, and can act in any of various ways to reduce or inhibit elevated Hh pathway activity when used in combination with cyclopamine. Further, the agent (e.g., an Hh pathway antagonist) can be administered in any way typical of an agent used to treat the particular type of prostate tumor or under conditions that facilitate contact of the agent with the target tumor cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell.

An agent useful in a method of the invention can be administered to the site of the prostate tumor, or can be administered by any method that results in the agent contacting the target tumor cells. Generally, the agent is formulated in a composition (e.g., a pharmaceutical composition) suitable for administration to the subject, which can be any vertebrate subject, including a mammalian subject (e.g., a human subject). Such formulated agents are useful as medicaments for treating a subject suffering from a prostate tumor that is characterized, in part, by elevated or abnormally elevated Hi pathway activity.

The terms “administration” or “administering” is defined to include an act of providing a compound of the invention or pharmaceutical composition to the subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The antagonists of the invention may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The agent, which acts as an Hh pathway antagonist to reduce or inhibit the elevated Eh pathway activity, can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest. 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of a composition containing the Hh pathway antagonist will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mnimic the structure of peptide domain; or based on a peptoid such as a vinylogous peptoid. Where the agent is a small organic molecule such as a steroidal alkaloid (e.g., cyclopamine), it can be administered in a form that releases the active agent at the desired position in the body (e.g., the stomach), or by injection into a blood vessel that the agent circulates to the target cells (e.g., prostate cells).

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

A composition containing an Hh pathway antagonist can be administered to an individual by various routes including, for example, orally or parenterally, such as intravenously, intramuscularly, subcutaneously, intraperitoneally, intrarectally, intracisternally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of the prostate tumor, for example, intravenously or intra-arterially into a blood vessel supplying a tumor.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the Hh pathway antagonist to treat a prostate tumor in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day which can be administered in single or multiple doses.

If desired, the effective daily dose of the active compound may be administered as two, three, four, five, six or more sub-doses administered separately at appropriate intervals throughout the day, optionally, in unit dosage forms. There may be a period of no administration followed by another regimen of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

When other therapeutic agents are employed in combination with the compounds of the present invention they may be used for example in amounts as noted in the Physician Desk Reference (PDR) or as otherwise determined by one having ordinary skill in the art.

The term “effective amount” is defined as the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality. For example, a “therapeutically effective amount” of; e.g., a Hh antagonist, with respect to the subject method of treatment, refers to an amount of the antagonist in a preparation which, when applied as part of a desired dosage regimen brings about, e.g., a change in the rate of cell proliferation and/or the state of differentiation and/or the rate of metastasis of a cell and/or rate of survival of a cell according to clinically acceptable standards for the disorder to be treated.

The term. “pharmaceutically acceptable” is defined as a carrier, whether diluent or excipient, that is compatible with the other ingredients of the formulation and not deleterious to the recipient thereof. The pharmaceutical composition of the invention can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The invention also provides a method of determining whether a prostate tumor of a subject is amenable to treatment with a Hh pathway antagonist as disclosed herein. The method can be performed, for example, by measuring the level Hh pathway activity in a prostate tumor cell sample of the tumor of a subject to be treated, and determining that Hh pathway activity is elevated or abnormally elevated as compared to the level of Hh pathway activity in corresponding normal cells, which can be a sample of normal (i.e., not tumor) cells of the subject having the tumor. Detection of elevated or abnormally elevated level Hh pathway activity in the tumor cells as compared to the corresponding normal cells indicates that the subject can benefit from treatment with an Hh pathway antagonist. A sample of cells used in the present method can be obtained using a biopsy procedure (e.g., a needle biopsy), or can be a sample of cells obtained by a surgical procedure to remove and/or debulk the tumor.

Elevated or abnormally elevated Hh pathway activity can be determined by measuring elevated expression of one or more (e.g., 1, 2, 3, or more) Hh pathway polypeptide(s), including, for example, one or more Hh ligands (e.g., SHH, IHH, and/or desert hedgehog), HF ligand receptors (e.g., PTCH), or transcription factors (a GLI family member), or a combination of such Hh pathway polypeptides. The elevated expression can be detected by measuring the level of a polynucleotide encoding the Hh pathway polypeptide (e.g., RNA) using, for example, a hybridization assay, a primer extension assay, or a polymerase chain reaction (PCR) assay (e.g., a reverse transcription-PCR assay); or by measuring the level the Hh pathway polypeptide(s) using, for example, an immunoassay or receptor binding assay. Alternatively, or in addition, elevated activity of one or more (e.g., 1, 2, 3, or more) Hh pathway polypeptide(s) can be determined. For example, elevated activity of Hh pathway transcription factor (e.g., a GLI family member) can be detected by measuring increased binding activity of the transcription factor to a cognate transcription factor regulatory element (e.g., using an electrophoretic mobility shift assay), or by measuring increased expression of a reporter gene comprising a cognate transcription factor regulatory element. Expression of an Hh pathway polypeptide having an inactivating mutation can be identified using, for example, an antibody that specifically binds to the mutant, but not to the normal (wild type), Hh polypeptide, wherein the mutation is associated with elevated Hh pathway activity. For example, common mutations that result in expression of an inactivated PTCH can define unique epitopes that can be targeted by diagnostic antibodies that specifically bind the mutant, but not wild type, PTCH protein.

The method of identifying a prostate tumor amenable to treatment with a Hh pathway antagonist can further include contacting cells of the sample with at least one Rh pathway antagonist, and detecting a decrease in Hh pathway activity in the cells following said contact. The decreased Rh pathway activity can be detected, for example, by measuring decreased expression of a reporter gene regulated by an Hh pathway transcription factor, or by detecting a decreased in proliferation or metastasis of the tumor cells. Such a method provides a means to confirm that the prostate tumor is amenable to treatment with an Rh pathway antagonist. Further, the method can include testing one or more different Hh pathway antagonists, either alone or in combination, thus providing a means to identify one or more Rh pathway antagonists useful for treating the particular prostate tumor being examined. Accordingly, the present invention also provides a method of identifying an agent useful for treating a prostate tumor having elevated Hh pathway activity.

The method of identifying an agent useful for treating a prostate tumor provides a means for practicing personalized medicine, wherein treatment is tailored to a patient based on the particular characteristics of the prostate tumor in the patient. The method can be practiced, for example, by contacting a sample of cells of a prostate tumor with at least one test agent, wherein a decrease in Rh pathway activity in the presence of the test agent as compared to Hh pathway activity in the absence of the test agent identifies the agent as useful for treating the prostate tumor. The sample of cells examined according to the present method can be obtained from the subject to be treated, or can be cells of an established prostate tumor cell line of the same type of tumor as that of the patient. In one aspect, the established prostate tumor cell line can be one of a panel of such cell lines, wherein the panel can include different cell lines of the same type of tumor and/or different cell lines of different tumors. Such a panel of cell lines can be useful, for example, to practice the present method when only a small number of tumor cells can be obtained from the subject to be treated, thus providing a surrogate sample of the subject's tumor, and also can be useful to include as control samples in practicing the present methods.

The present methods can be practiced using test agents that are known to be effective in treating a prostate tumor having elevated Hh pathway activity (e.g., a steroidal alkaloid such as cyclopamine or jervine; and/or other SMO antagonist such as SANT-1 or SANT-2; and/or an anti-Hh ligand antibody such as an anti-SHH antibody) in order to identify one or more agents that are particularly useful for treating the prostate tumor being examined, or using test agents that are being examined for effectiveness. In addition, the test agent(s) examined according to the present method can be any type of compound, including, for example, a peptide, a polynucleotide, a peptidomimetic, or a small organic molecule, and can be one or a plurality of similar but different agents such as a combinatorial library of test agents, which can be a randomized or biased library or can be a variegated library based on known effective agent such as the known Hh pathway antagonist, cyclopamine (see, for example, U.S. Pat. No. 5,264,563; and U.S. Pat. No. 5,571,698, each of which is incorporated herein by reference). Methods for preparing a combinatorial library of molecules, which can be tested for Hh pathway antagonist activity, are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markdand et al., Gene 109:13-19, 1991; each of which is incorporated herein by reference); a peptide library (U.S. Pat. No. 5,264,563, which is incorporated herein by reference); a peptidomimetic library (Blondelle et al., supra, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference; each of which is incorporated herein by reference); an oligosaccharide library (York et al., Carb. Res. 285:99-128, 1996; Liang et al., Science 274:1520-1522, 1996; Ding et al., Adv. Expt. Med. Biol. 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents, (Gordon et al., J. Med. Chem. 37:1385-1401, 1994; Ecker and Crooke, supra, 1995; each of which is incorporated herein by reference).

The method of identifying an agent useful for treating a prostate tumor having elevated Hh pathway activity can performed by contacting the sample of cells ex vivo, for example, in a culture medium or on a solid support. Alternatively, or in addition, the method can be performed in vivo, for example, by transplanting a tumor cell sample into a test animal (e.g., a nude mouse), and administering the test agent to the test animal. An advantage of the in vivo assay is that the effectiveness of a test agent can be evaluated in a living animal, thus more closely mimicking the clinical situation. Since in vivo assays generally are more expensive, the can be particularly useful as a secondary screen, following the identification of “lead” agents using an in vitro method.

When practiced as an in vitro assay, the methods can be adapted to a high throughput format, thus allowing the examination of a plurality (i.e., 2, 3, 4, or more) of cell samples and/or test agents, which independently can be the same or different, in parallel. A high throughput format provides numerous advantages, including that test agents can be tested on several samples of cells from a single patient, thus allowing, for example, for the identification of a particularly effective concentration of an agent to be administered to the subject, or for the identification of a particularly effective agent to be administered to the subject. As such, a high throughput format allows for the examination of two, three, four, etc., different test agents, alone or in combination, on the cells of a subject's prostate tumor such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test agent(s); or two or more samples of cells obtained from one subject can be examined with same or different amounts of one or a plurality of test agent. In addition, cell samples, which can be of the same or different subjects, can be examined using combinations of test agents and/or known effective agents. Variations of these exemplified formats also can be used to identifying an agent or combination of agents useful for treating a prostate tumor having elevated Hh pathway activity.

When performed in a high throughput (or ultra-high throughput) format, the method can be performed on a solid support (e.g., a microtiter plate, a silicon wafer, or a glass slide), wherein samples to be contacted with a test agent are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for Hh pathway activity and/or cell viability.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Ligand Stimulated hedgehog Pathway Activity is Associated with Growth and Metastasis of Prostate Tumors

The following example demonstrates that prostate tumors display elevated Hh pathway activity, and that cyclopamine, a Hh pathway antagonist, can decrease the elevated Hh pathway activity and inhibit proliferation and/or metastasis of the prostate cancer cells.

It was shown that primary cells and cell lines from metastatic but not localized prostate tumors displayed endogenous ligand-stimulated Hedgehog (Hh) pathway activity, and that Hh pathway blockade produces complete and durable regression of metastasis-derived human prostate cancer xenografts. It was also shown that Hedgehog pathway activity is required for regeneration of prostate epithelium in rodent castrates, suggesting a requirement for pathway activity in similar proliferative progenitor cell populations in the regenerating organ and in metastatic tumors. Furthermore, cyclopamine inhibition-of Hh pathway activity blocks lethality in mice of a highly metastatic prostate tumor, whereas over-expression of Gli, a transcriptional effector of the Hh pathway, converts relatively indolent tumor cells to a rapidly lethal metastatic phenotype. Hh pathway activity was found to dramatically increase invasiveness of prostate cancer cells and promote changes in expression of genes known to modulate metastasis. The role of Hh pathway activity in promoting metastatic growth suggests that pathway antagonists may offer significant therapeutic improvements in the treatment of advanced protate cancer.

### A. Cells and Tissues

PC3, CWR22RV1, DUI45 and LnCAP (American Tissue Type Collection, Manassas, Va.) cells, were cultured in growth media (RPMI-1640 supplemented with 10% fetal bovine serum). AT6.3 and AT2.1 cells were cultured in growth media supplemented with 250 nM dexamethasone. Prostate Epithelial cells (PrE; Cambrex Biochemicals, Walkersville, Md.) were cultured according to vendor's instructions. Tissues samples are described in Table 1.

TABLE 1
Normal and tumor tissue obtained from patients
undergoing prostatectomy

| Normal | | | | | Tumor in |
|---|---|---|---|---|---|
| (sample | Cancer | Pathologic | Gleason | Tumor at | Sample |
| no.)* | (sample no.) | stage | Score | Surgical | (%)* |
| 1 | 1 | TX N0 | 6 | Yes | 75 |
| 2 | 2 | TX N0 | 6 | Yes | 85 |
| 3 | 3 | T2 N0 | 6 | No | 85 |
| 4 | 4 | T2 N0 | 6 | No | 85 |
| 5 | 5 | T2 N0 | 6 | No | 15 |
| 6 | 6 | T3a N0 | 7 | No | 90 |
| 7 | 7 | T2 N0 | 5 | No | 90 |
| 8 | 8 | T2 N0 | 6 | No | 5 |
| 9 | 9 | T3a N0 | 7 | No | 95 |
| 10 | 10 | T3a N0 | 7 | No | 95 |
| 11 | | T3b N1 | 7 | No | n/a |
| 12 | | T3b N0 | 7 | Yes | n/a |
| | 13 | T2 N0 | 6 | No | 85 |
| | 14 | T2 N0 | 7 | No | 85 |

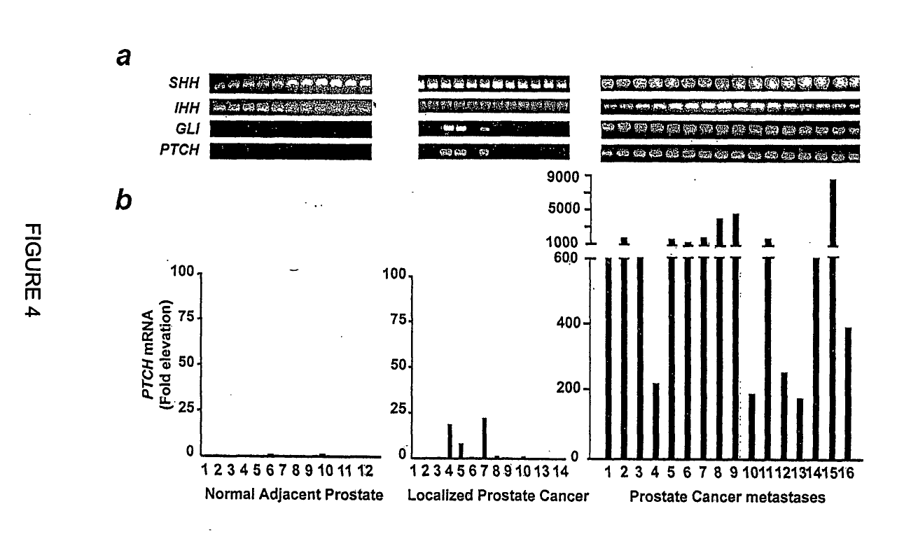

*Sample numbers refer to FIG. 4. Samples 1-10 are matched normal-tumor pairs, each from a single patient. A single tissue block was selected from each case and used to prepare histologic sections and total cellular RNA. Sections were scored by a genitourinary pathologist (D.M.B.) for percentage of sample involved by tumor.

TABLE 2
Sites of metastasis sampled from 12 prostate cancer patients at autopsy.

| No*. | Site |
|---|---|
| 1 | L. Adrenal |
| 2 | Hilar LN |
| 3 | R ObturatorLN |
| 4 | Liver |
| 5 | Mesenteric LN |
| 6 | Diaphragm |
| 7 | Obturator LN |
| 8 | Liver |
| 9 | Subdural space |
| 10 | Rib |
| 11 | Vertebra |
| 12 | Axillary node |
| 13 | Mediastinal LN |
| 14 | Axillary LN |
| 15 | Para-aortic LN |
| 16 | Subdural space |

*Sample numbers refer to FIG. 4

### B. RNA Isolation and Analysis:

Total cellular RNA was isolated and used to synthesize random primed first strand cDNA for analysis by conventional and quantitative real time (SYBR green) PCR (qRT-PCR) as described (Berman, D. M. et al. Medulloblastoma growth inhibition by hedgehog pathway blockade. Science 297, 1559-61 (2002)). Amplification of H pathway components was normalized in qRT-PCR experiments to that of endogenous phosphoglycerate kinase in each sample. Oligonucleotide primers used in quantitative reeal-time and conventional amplification of reverse transcribed mRNA (RT-PCR) are shown in Table 4. The specificity of each primer pair was confirmed by sequencing amplified products.

### C. Reporter Assays

Subconfluent triplicate cultures of cells plated in 96-well plates were transfected with 100 ng DNA per well of control Renilla luciferase reporter (pRL-SV40, Promega, Madison, Wis.) (5% w/w DNA) and the Gli-luciferase reporter (95% w/w DNA) using Fugene 6 transfection reagent at a 3:1 ratio (v/w) of reagent to DNA. After 48 hours media was replaced and supplemented with 5E1 antibody, recombinant doubly lipid modified Sonic Hedgehog (ShhNp) protein (Taipale, J. et al. Effects of oncogenic mutations in Smoothened and Patched can be reversed by cyclopamine. Nature 406, 1005-9. (2000)), cyclopamine or tomatidine at the concentrations indicated in the accompanying figure legends and incubated for an additional 48 hours. Lysates were prepared and reporter activity was measured using the Dual Luciferase assay system (Promega, Madison, Wis.) according to the manufacturer's protocol. In all assays, Gli-luciferase levels were normalized to control Renilla luciferase levels.

### D. Stable Transfections

Cells were transfected in 100 mm dishes with 15 μl of Fugene 6 transfection reagent (Roche, Indianapolis, Ind.) and 5 μg of plasmid DNA, consisting of pKO-Neo (Invitrogen,Carlsbad, Calif.) alone or in a 1:19 ratio with either pSRa-FLAG-Glil or pSRa-FLAG-GlilZFD (Park, H. L. et al. Mouse Gli1 mutants are viable but have defects in SHH signaling in combination with a Gli2 mutation. Development 127, 1593-605. (2000)).Transfectants were selected with Geneticin (200 μg/ml; Gibco, Grand Island, N.Y.) and subdloned.

### E. Viability Assays

Viable cell mass, (reduction of an aqueous soluble tetrazolium salt to form a coloured product) was assayed using the CellTiter96 reagent (Promega, Madison, Wis.) as described (Berman, D. M. et al. Medulloblastoma growth iriibition by hedgehog pathway blockade. Science 297, 1559-61 (2002)).

### F. Xenografts

CWR22RV1 (n=14) and PC3 tumor xenografts (n=20) were grown by injecting 0.1 ml of Hanks Balanced Salt Solution and Matrigel (1:1) (Beckton Dickinson, Franklin Lakes, N.J.) containing 2.5×10⁶ cells subcutaneously at each of two locations (right anterior and posterior flank) per athymic mouse. In one experiment, groups of animals bearing tumors with an average volume (length×width×0.5×[length+width]) of 411 mm³ and 502 mm³ were treated with 0.1 ml vehicle (triolein: ethanol 4:1 vol./vol.) alone, or with cyclopamine (10 mg/kg/day) injected subcutaneously into the animal's left dorsum daily for 9 (PC3) or 10 (CWR22RV1) days. Animals were euthanized and tumors harvested for Ki-67 staining. In a second experiment, CWR22RV1 (n=20), CWR22RV1 GLI (n=8) and PC3 (n=12) tumors were grown to an average volume of 195 mm³ and treated with 50 mg/kg/day cyclopamine or vehicle. Treatment was stopped after 28 days (PC3) or 22 days (22RV1), 7 days after all tumors appeared to have completely regressed. AT6.3, AT 2.1 and AT2.1-GLI rat prostate cancer cells in PBS were injected as above but without Matrigel in athymic mice and treatment was commenced the next day with daily injections of either intraperitoneal cyclopamine at two doses—10 mg/kg/day or 50 mg/kg/day(AT 6.3; n=12), subcutaneous cyclopamine at 50 mg/kg/day (AT2.1; n=5), (AT6.3; n=5) or corn oil-vehicle (Sigma, St. Louis, Mo.) alone (AT2.1; n=5), (AT6.3; n=6), (AT2.1-GLI; n=5). Mice were observed daily for distress and experiments were carried out according to approved institutional protocols. Individual tumor volumes were plotted and regression curves were generated using analysis software to determine individual tumor growth rates.

### G. Prostate Regeneration

C57B16/J mice (Jackson labs) were castrated (standard surgical procedures, scrotal route), rested for 7 days, and treated with daily subcutaneous injections of vehicle (80% glycerol trioleate in ethanol) alone, with dihydrotestosterone (DHT; 50 mg/kg), or with DHT and cyclopamine (50 mg/kg) for 10 days. Prostates were collected, weighed, and processed for histology.

### H. In Vitro Invasion Assays

Cells were pre-treated with either 3 μM Cyclopamine or 3 μM tomatidine for a period of 24 hours, trypsinized, and 2×10⁵ cells were dispensed into the top chambers of a 24 well-Matrigel invasion chamber assay plate (BD Biocoat; Becton-Dickenson, Bedford Mass.). Cells reaching the lower chamber were counted according to the manufacturer's protocol. Results were normalized to viable cell mass assayed as described above.

### I. Ki-67 Staining

Sections prepared from control- and cyclopamine-treated tumors were pre-treated as described (Berman (2001), supra) and incubated with rabbit polyclonal antisera against Ki-67 (NovoCastra, Burlingame, Calif.). Immunodetection was performed with the VectaStain ABC kit (Vector Laboratories; Burlingame, Calif.) according to the manufacturer's instructions. The proliferation index was calculated as the ratio of Ki-67-positive to Ki67 negative nuclei in at least 300 cells examined in each of 5 randomly selected regions.

Expression of Hh pathway ligands and endogenous targets in several widely studied human prostate cancer cell lines provides information regarding the potential role and mechanism of pathway activation in the biology of the common prostate tumor. Pathway activity can be monitored by measuring levels of mRNA encoding the pathway components GLI and PATCHED (PTCH, the target of Gorlin Syndrome mutations). Both GLI and PTCH are transcriptional targets of pathway activation with opposite roles in pathway response, with GLI serving as a positive transcriptional effector and PTCH functioning to restrain pathway activity by suppressing the action of Smoothened (SMO). This negative function of PTCH is blocked by binding of Hh ligand, thus permitting pathway activation via SMO (Taipale (2001), supra; Ingham, P. W. & McMahon, A. P. Hedgehog signaling in animal development: paradigms and principles. Genes Dev. 15, 3059-87 (2001)).

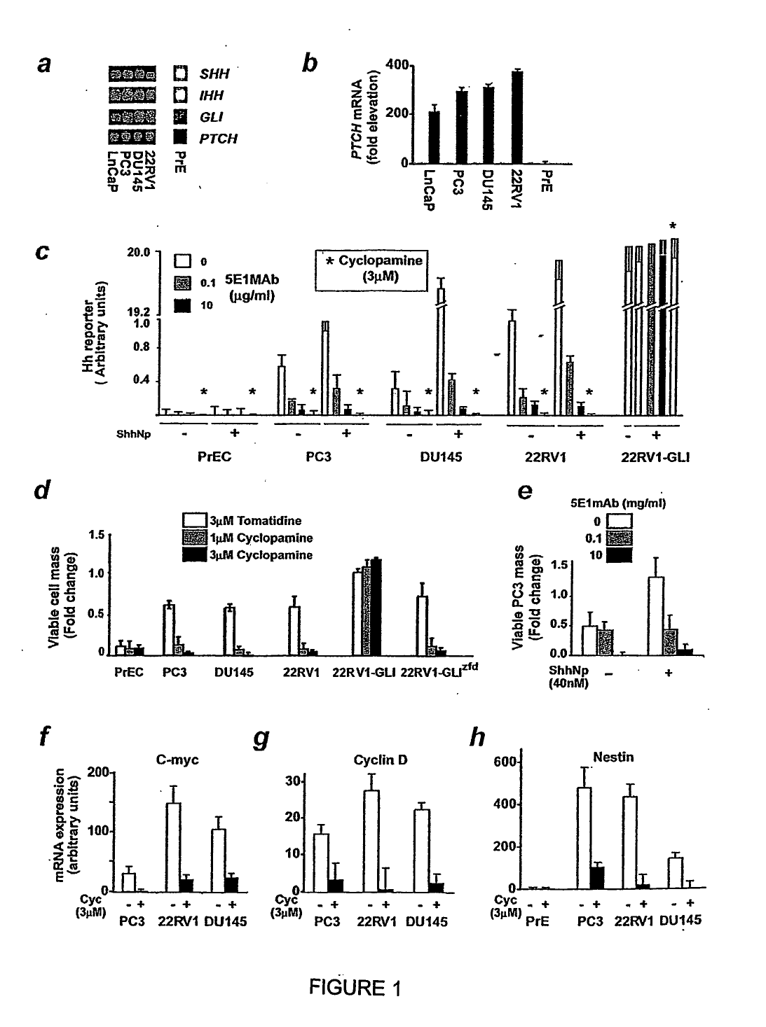

Four tumor-derived cell lines were examined (PC3, DU145, CWR22RV1, LnCAP) and found to express transcripts encoding Soinic (SHH) and Iildian (IHH) hedgehog ligands, as do benign prostate epithelial cells (PrE; FIG. 1a). Tumor cells but not PrE cells also express PTCH and GLI transcripts, suggesting that the Hh pathway is specifically activated in tumor cells. In confirmation of this active state, quantitative RT-PCR analysis revealed that levels of PTCH message were ˜200-400 fold elevated in cancer cells relative to benign PrE cells (FIG. 1b). We also noted high luciferase activity in tumor cells upon introduction of a Hh-responsive GLI-luciferase reporter (FIG. 1c) (se also, Taipale, J. et al. Effects of oncogenic mutations in Smoothened and Patched can be reversed by cyclopamine. Nature 406, 1005-9. (2000)). This activity was fully suppressible by treatment with cyclopamine, which specifically inhibits Hh pathway response by binding to and stabilizing the inactive conformation of SMO (Taipale (2000), supra; Cooper, M. K., Porter, J. A., Young, K. E. & Beachy, P. A. Plant-derived and synthetic teratogens inhibit the ability of target tissues to respond to Sonic hedgehog signaling. Science 280, 1603-1607 (1998); Incardona, J. P., Gaffield, W., Kapur, R. P. & Roelink, H. The teratogenic Veratrum alkaloid cyclopamine inhibits sonic hedgehog signal transduction. Development 125, 3553-3562 (1998); and Chen, J. K., Taipale, J., Cooper, M. K. & Beachy, P. A. Inhibition of Hedgehog signaling by direct binding of cyclopantine to Smoothened. Genes Dev. 16, 2743-8 (2002)). As seen in 22RV1-GLI cells, cyclopamine blockade of SMO was bypassed by stable overexpression of GLI, demonstrating the specificity of the cyclopamine effect in the Hh pathway.

Constitutive reporter activity in prostate cancer cells could be augmented by addition of exogenous Shh ligand (ShhNp), and both endogenous and exogenously augmented activities were blocked in a dose dependent manner by treatment with a monoclonal antibody (5E1) that neutralizes Ihh and Shh ligands (FIG. 1c) (see also, Wang, L. C. et al. Regular articles: conditional disruption of hedgehog signaling pathway defines its critical role in hair development and regeneration. J. Invest. Dermatol. 114, 901-8 (2000); Ericson, J., Morton, S., Kawakami, A., Roelink, H. & Jessell, T. M. Two critical periods of Sonic Hedgehog signaling required for the specification of motor neuron identity. Cell 87, 661-73 (1996)). Thus, although endogenous ligand expression in these tumor-derived cells produces significant pathway activity, this activity is further enhanced by exogenous ligand stimulation. The benign PrE cells, despite expression of SHH and IHH transcripts, did not display constitutive Hh pathway activity and failed to respond to exogenously added ligand, suggesting that Hh-responsiveness constitutes a significant difference between benign and malignant prostate epithelial cells.

Having established the responsiveness of transcription in prostate cancer cells to stimulation with endogenous and exogenous Hh ligand, the effects of pathway blockade on growth were examined. Treatment with cyclopamine dramatically inhibited growth of PC3, DU145 and 22RV1 cells (FIG. 1d), as compared to treatment with the structurally related but inactive compound, tomatidine (Cooper (1998), supra; Incardona, (1998) supra). Pathway specificity in this anti-proliferative effect of cyclopamine again was demonstrated through bypass of cyclopamine blockade with over-expression of GLI, but not of GLI^{zfd} (FIG. 1d), which lacks the zinc finger DNA-binding domain of GLI and consequently is transcriptionally inert (Park, H. L. et al. Mouse Gli1 mutants are viable but have defects in SHH signaling in combination with a Gli2 mutation. Development 127, 1593-605. (2000)). Pathway specificity of this inhibitory growth effect was further confirmed in PC3 cells by treatment with the neutralizing antibody, 5E1 (FIG. 1e). As molecular correlates of cell growth inhibition by pathway blockade, quantitative RT-PCR showed that cyclopamine treatment reduced expression of transcripts encoding c-myc and cyclin DI (FIG. 1f,g), which promote GI cell cycle transition and have been implicated in prostate cancer growth (Fleming, W. H. et al. Expression of the c-myc protooncogene in human prostatic carcinoma and benign prostatic hyperplasia. Cancer Res. 46, 1535-8 (1986); Ellwood-Yen, K. et al. Myc-driven murine prostate cancer shares molecular features with human prostate tumors. Cancer Cell 4, 223-38 (2003); and Aaltomaa, S., Lipponen, P., Eskelinen, M., Ala-Opas, M. & Kosma, V. M. Prognostic value and expression of p2l (wafl/cipl) protein in prostate cancer. Prostate 39, 8-15(1999)).

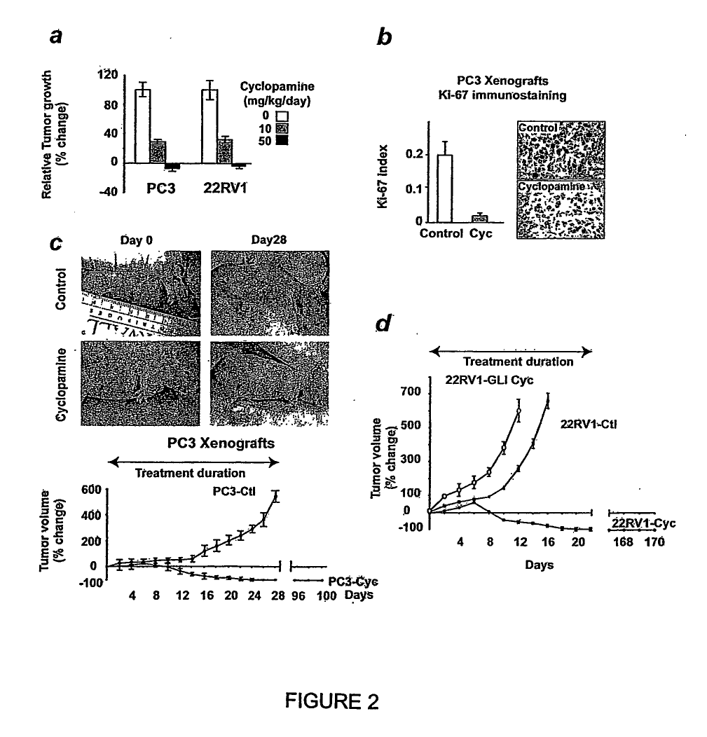

Because requirements for proliferation of cells cultured in vitro could differ from those for the growth of established tumors in vivo, the role of Hh pathway activity was tested by establishing subcutaneous PC3 and 22RV1 xenograft tumors in athymic mice. Tumors were inoculated and allowed to reach a median size of 155 mm³ after an average of 16 days of growth before initiation of daily treatment with subcutaneous injections of cyclopamine (10 or 50 mg/kg) or vehicle alone. By the ninth day of treatment suppression of tumor growth at 10 mg/kg cyclopamine was observed, and actual regression of tumors at 50 mg/kg (FIG. 2a). Animals treated at the intermediate dose of 10 mg/kg were sacrificed and a 90% reduction in staining for the proliferation antigen Ki67 was noted (FIG. 2b), consistent with the reduced but incompletely suppressed growth of these tumors in vivo. Animals that began treatment at the higher dose continued to receive 50 mg/kg, and displayed complete regression of the tumors within 20-24 days of treatment (FIG. 2c,d). Notably, this effect was durable, as cessation of treatment did not result in regrowth of tumors, even after observation periods of 86 days (PC3) and 170 days (22RV1) (FIG. 2c,d). As seen in vitro, xenograft tumors from 22RV1 cells overexpressing GLI were not affected by cyclopamine treatment, and actually grew faster than vehicle-treated tumors (FIG. 2d). The ability of GLI overexpression to bypass the cyclopamine effect in vivo reinforces the finding that cyclopamine suppression of tumor growth is mediated specifically by Hh pathway blockade. Furthermore, the acceleration of tumor growth by GLI overexpression confirms in vivo that the rate of tumor cell growth corresponds to the degree of Hh pathway activity (FIGS. 1c,d; 2d).

Complete and durable tumor regression like that produced by cyclopamine treatment has not been reported previously for any other pharmacologic agent in experimental models of human prostate cancer, and this result suggests that cells capable of renewing the tumor, i.e., of functioning as tumor progenitors or stem cells (Al-Hajj, M., Wicha, M. S., Benito-Hemandez, A., Morrison, S. J. & Clarke, M. F. Prospective identification of tumorigenic breast cancer cells. Proc. Natl. Acad. Sci. USA 100, 3983-8 (2003); Singh, S. K. et al. Identification of a cancer stem cell in human brain tumors. Cancer Res. 63, 5821-8 (2003); and Kondo, T., Setoguchi, T. & Taga, T. Persistence of a small subpopulation of cancer stem-like cells in the C6 glioma cell line. Proc. Natl. Acad. Sci. USA 101, 781-6 (2004)), require Hh pathway activity for their maintenance. Cyclopamine also suppressed transcription of the gene encoding Nestin, an intermediate filament protein whose expression has not been described previously in the prostate, but which marks progenitor cell populations in other endodermally-derived and neural tissues (Esni, F., Staffers, D. A., Takeuchi, T. & Leach, S. D. Origin of exocrine pancreatic cells from nestin-positive precursors in developing mouse pancreas. Mech. Dev. 121, 15-25 (2004); Kachinsky, A. M.) Dominov, J. A. & Miller, J. B. Myogenesis and the intermediate filament protein, nestin. Dev. Biol. 165, 216-28 (1994); Lendahl, U., Zimmerman, L. B. & McKay, R. D. CNS stem cells express a new class of intermediate filament protein. Cell 60, 585-95 (1990); and Zulewski, H. et al. Multipotential nestin-positive stem cells isolated from adult pancreatic islets differentiate ex vivo into pancreatic endocrine, exocrine, and hepatic phenotypes. Diabetes 50, 521-33 (2001))(FIG. 1h).

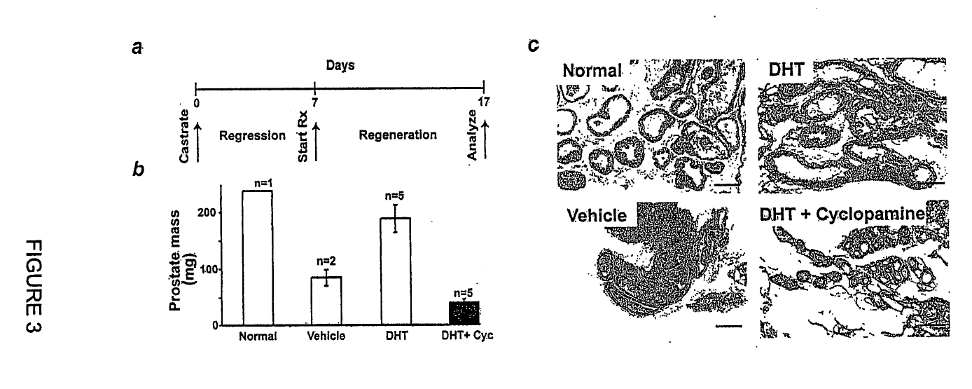

To further investigate the role of Hh pathway activity in progenitor cell homeostasis, epithelial regeneration in rodent prostates was examined using castration-induced androgen withdrawal as a well-established method for ablation of prostate epithelium (Moore, R. J. & Wilson, J. D. The effect of androgenic hormones on the reduced nicotinamide adenine dinucleotide phosphate:delta-4-3-ketosteroid 5 alpha-oxidoreductase of rat ventral prostate. Endocrinology 93, 581-92 (1973); English, H. F., Santen, R. J. & Isaacs, J. T. Response of glandular versus basal rat ventral prostatic epithelial cells to androgen withdrawal and replacement. Prostate 11, 229-42 (1987)). Following seven days of androgen withdrawal, which dramatically reduces epithelial content (by>90%) and is thought to leave a population greatly enriched in progenitor cells (English, et al., (1987) supra; Meeker, A. K., Sommerfeld, H. J. & Coffey, D. S. Telomerase is activated in the prostate and seminal vesicles of the castrated rat. Endocrinology 137, 5743-6 (1996)), a ten day course of androgen supplementation (dihydrotestosterone; DHT, 50 mg/kg/d) resulted in re-growth of prostate of nearly normal size (FIG. 3b) and histological appearance (i.e., large complex glands lined with tall columnar epithelium) (FIG. 3b,c). In sharp contrast, however, cyclopamine blockade abolished prostate regeneration (FIG. 3b,c), yielding small, simple, atrophic glands lined with low cuboidal epithelium, similar in appearance to prostates in vehicle-treated castrates (FIG. 3c). The inhibitory effects of cyclopamine blockade in regeneration of prostate epithelium and in tumor growth may reflect a common requirement for Hh pathway activity in expansion of similar pools of proliferative progenitor cells.

As prostate cancer cell lines are established from tumor metastasis and furthermore undergo some degree of selection during adaptation to long-term proliferation in vitro, it was important to more directly assess the status of Hh pathway activity in localized as well as metastatic prostate cancer. Therefore, samples of lethal metastasis harvested at autopsy as well as samples of localized tumors and adjacent normal tissue from radical prostatectomies were examined. By RT-PCR all samples of normal and localized or metastatic malignant prostate tissue were found to express SHH and IHH (FIG. 4a). However, all metastatic tumors (n=16 samples from 13 patients) but no benign prostate samples (n=12 histologically verified normal tissue samples) expressed Hh pathway targets PTCH and GLI (FIG. 4a), suggesting an active state of the pathway in metastatic tumors but not in benign prostate tissue (FIG. 3A). Of considerable interest, however, only 3 of 12 samples from localized malignancies expressed PTCH and GLI, and quantitative RT-PCR analysis (FIG. 4b) further revealed that PTCH mRNTA levels in these three samples never exceeded one-tenth that noted in the lowest-expressing metastatic tumors. This dramatic disparity in endogenous PTCH expression indicates that the state of Hh pathway activity is strongly correlated with metastasis.

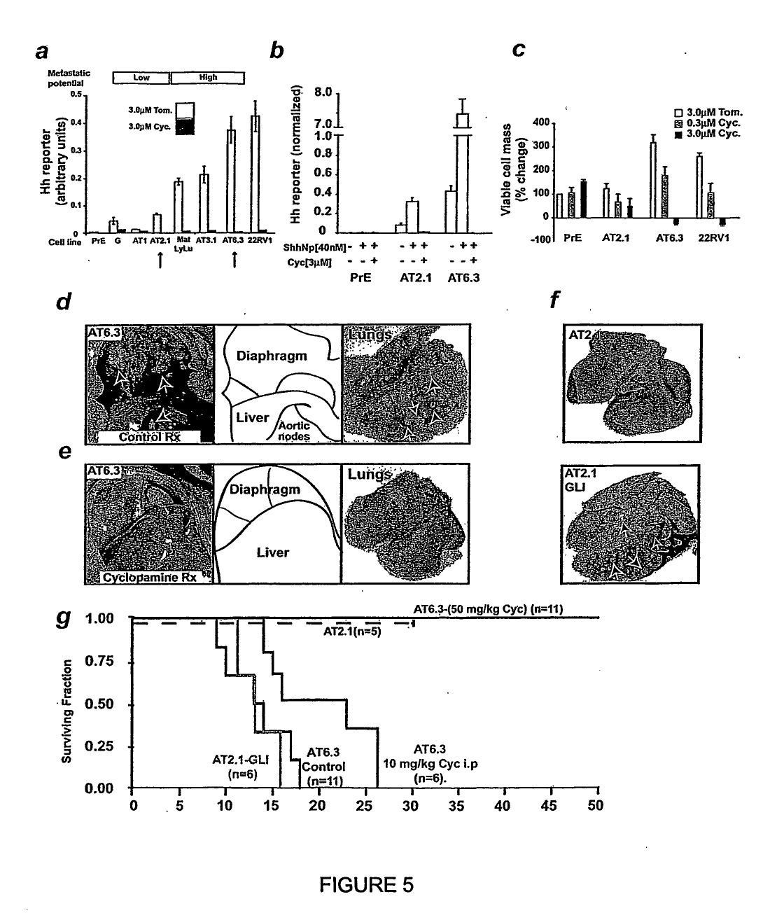

The role of Hh pathway activity in metastasis suggested by these findings was then explored. However, as human prostate cancer xenografts metastasize slowly and infrequently in mouse models, a series of rodent cell lines established from tumors with widely varying metastatic potential (Isaacs, J. T., Isaacs, W. B., Feitz, W. F. & Scheres, J. Establishment and characterization of seven Dunning rat prostatic cancer cell lines and their use in developing methods for predicting metastatic abilities of prostatic cancers. Prostate 9, 261-81(1986); Dong, J. T. et al. KAI1, a metastasis suppressor gene for prostate cancer on human chromosome 1 1p11.2. Science 268, 884-6 (1995)) was used. These lines all derive ultimately from a single parental tumor, the Dunning R3327 rat prostate cancer model (Dunning, W. F. Prostate Cancer in the Rat. Natl. Cancer Inst. Monogr. 12, 351-69 (1963)), but were selected during serial passage in vivo according to their ability to metastasize. Interestingly, of six cell lines surveyed for pathway activity using the Gli-luciferase reporter, the three derived from tumors characterized as highly metastatic (Mat-LyLu, AT3.1, and AT6.3) displayed relatively high levels of pathway activity, comparable to those in human prostate cancer cell lines (FIG. 5a), whereas the three lines from tumors characterized as displaying little or no metastatic ability (G, AT1, and AT2.1) displayed only low levels of pathway activity, albeit somewhat higher than that observed in benign PrE cells (FIG. 5a). These results further support an association between endogenous Hh ligand-stimulated pathway activation and metastatic potential.

A single cell line each from the high (AT6.3) and low (AT2.1) metastasis group was selected for further characterization. The AT6.3 cell line (high metastasis group) was particularly responsive to addition of exogenous ShbNp ligand (FIG. 5b), and furthermore was as sensitive in its growth as human prostate cancer cell lines to Ih pathway blockade by cyclopamine and 5E1 neutralizing antibody (FIG. 5c and data not shown). Subcutaneous inoculation of AT6.3 cells in nude mice confirmed their previous characterization as highly metastatic, with extensive and macroscopically visible metastatic colonization of visceral organs in the thoracic and abdominal cavities (FIG. 5d). These mice invariably die within a few weeks of inoculation (FIG. 5g). The AT2.1 cells, previously characterized as displaying low metastatic ability, produced no mortality and no evidence of metastasis 30 days after subcutaneous inoculation (FIG. 5f,g).

An ATp2.1-GLI cell line stably transfected for overexpression of the Hh pathhway effector GLI was then established. Whereas mice bearing subcutaneous tumors from parental AT2.1 cells all survived throughout the 30 day observation period, mice inoculated subcutaneously with AT2.1-GLI cells all died within 16 days (n=6), comparable to the 18 day maximal survival of mice (n=11) inoculated with AT6.3 cells (FIG. 5f,g). Remarkably, as also noted for the AT6.3 cells, AT2.1-GLI cells produced widespread visceral metastasis (FIG. 5, and data not shown), and activation of Hh pathway targets thus appears sufficient for conferral of a lethal metastatic phenotype.

Having established the sufficiency of transcriptional activation of Hh pathway targets for conversion of AT2.1 cells to a lethal metastatic phenotype, the ability of metastatic phenotype of AT6.3 cells to be reversed by cyclopamine blockade of Hh pathway activity was determined. This analysis is complicated by the fact that cyclopamine treatment blocks tumor growth altogether, as noted in vitro and upon subcutaneous injection of cyclopamine (50 mg/kg/day) into mice inoculated with AT6.3 cells (data not shown). To more specifically address tumor metastasis, these studies were repeated with an intraperitoneal cyclopamine treatment regimen. This route of administration at 10 or 50 mg/kg/day permitted growth of subcutaneous AT6.3 tumors, but inhibited metastasis and improved survival (FIG. 5e,g). The intermediate 10 mg/kg/day dose thus increased median survival to 19 days with all animals dead by 26 days, and the 50 mg/kg/day dose blocked metastasis (FIG. 5e) and prevented death throughout a 50 day treatment period (FIG. 5g).

Although the primary AT6.3 subcutaneous tumors continued to grow under both the 50 and 10 mg/kg/day intraperitoneal treatment regimen, the rate of growth was reduced from that of vehicle treated tumors (26.2, 9.2 and 4.9%/o/day respectively for untreated, 10 mg/kg/day, and 50 mg/kg/day cyclopamine; Table 3). In addition, conversion of AT2.1 to a metastatic phenotype by overexpression of GLI also increased growth rate (from 3.4 to 33.7%/o/day), raising the possibility that growth rate may determine metastatic potential. As a potential indicator of metastatic behavior that can be assayed independently of growth, the invasiveness of cells in modified Boyden chamber assays, which utilize a chamber separated by a collagen-coated membrane with 8 micron pores was examined. Invasive cells with the ability to penetrate the matrix can migrate and adhere to the side of the membrane opposite that on which they are seeded, and such behavior correlates with metastatic potential in vivo (Albini, A. et al. A rapid in vitro assay for quantitating the invasive potential of tumor cells. Cancer Res. 47, 3239-45 (1987); Guan, R. J. et al. Drg-1 as a differentiation-related, putative metastatic suppressor gene in human colon cancer. Cancer Res. 60, 749-55 (2000); and Cano, A. et al. The transcription factor snail controls epithelial-mesenchymal transitions by repressing E-cadherin expression. Nat. Cell Biol. 2, 76-83 (2000)).

TABLE 3
Growth rates of subcutaneous tumors and median survival of mice after
inoculation of AT2.1, AT2.1-GLI and AT6.3 cells
and subsequent treatment.

| | | | | Growth rate |
|---|---|---|---|---|
| Tumor | | | Median | (% tumor |
| type | Treatment | n | Survival | volume/day) |
| AT2.1 | Vehicle | 5 | No death | 3.4 ± 0.53 |
| AT2.1 | Cyclopamine s.c. | 5 | No tumors | NA |
| AT2.1- | Vehicle | 5 | 13 days | 33.7 ± 3.04 |
| GLI | | | | |
| AT6.3 | Vehicle | 11 | 13.5 days | 26.3 ± 4.7 |
| AT6.3 | Cyclopamine i.p. (10 mg/kg) | 6 | 19 days | 9.2 ± 2.4 |
| AT6.3 | Cyclopamine i.p. (50 mg/kg) | 6 | No death | 4.9 ± 1.0 |
| AT6.3 | Cyclopamine s.c. | 5 | No tumors | NA |
| | (50 mg/kg) | | | |

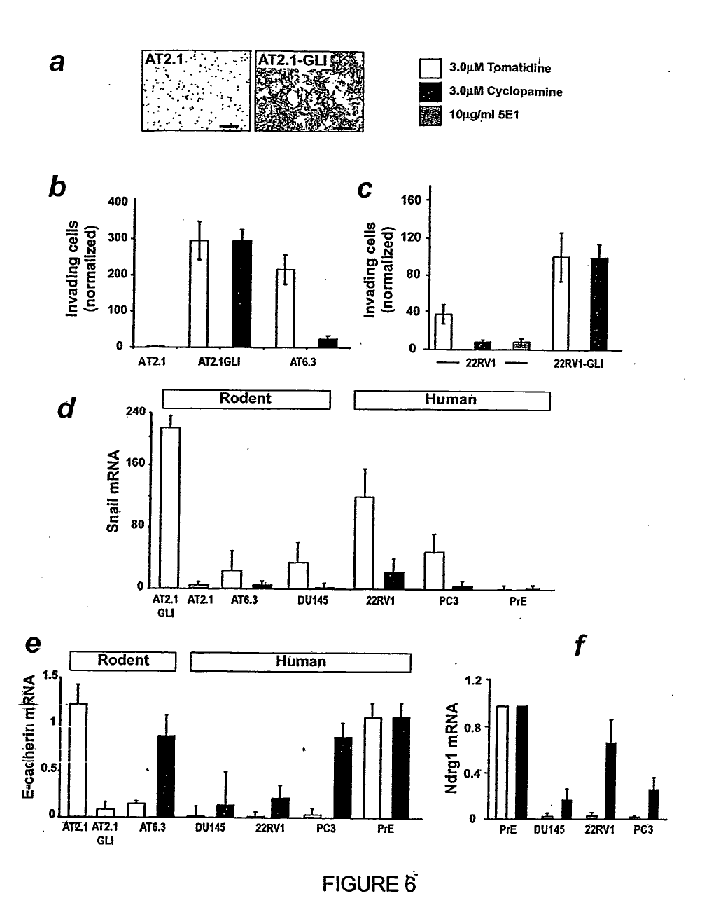

Consistent with the dramatic difference in metastatic ability between AT2.1 and AT2.1-GLI cells (FIG. 5f,g), AT2.1-GLI cells readily penetrate the matrix and populate the bottom surface of the membrane (the side opposite seeding), whereas AT2.1 cells rarely do so (FIG. 6a,b). By counting cells on the bottom of the membrane and normalizing to viable cell mass, it was noted that the GLI-overexpressing cells are approximately 125-fold more invasive than the parental cells (FIG. 6a,b). AT6.3 cells also displayed invasiveness comparable to that of AT2.1-GLI cells, and this invasiveness was reduced approximately nine-fold by treatment with cyclopamine (FIG. 6b). Cyclopamine treatment did not reduce the invasiveness of AT2.1-GLI cells, demonstrating a specific role for GLI-mediated transcription in Hh-dependent invasive behavior (FIG. 6b). The growth rate of cells is not a significant factor in these assays, as equal numbers of cells were incubated for 20 hours and the number of invading cells at the end of the experiment was normalized to the total viable cell mass on both sides of the membrane.

Having established that Hh-dependent changes in invasive behavior can be distinguished from cell growth, the transcription of genes whose regulation may specify cellular properties that confer invasive character was examined. In general, metastasis-associated invasiveness of epithelial tumors is thought to involve a transition to greater mesenchymal character (Cano (2000), supra; Birchmeier, C, Birchmeier, W., Gherardi, E. & Vande Woude, G. F. Met, metastasis, motility and more. Nat. Rev. Mol. Cell Biol. 4, 915-25 (2003)). Such transitions, both in normal development and in metastasis, are associated with expression of the transcription factor Snail (Cano (2000), supra). Snail acts in part by suppressing expression of proteins important in maintenance of epithelial organization, such as E-cadherin (Cano (2000), supra). We found that GLI expression in AT2.1 cells dramatically stimulated the expression of Snail mRNA (FIG. 6d). Snail expression in AT6.3 cells in contrast is constitutive, and can be suppressed by treatment with cyclopamine (FIG. 6d). As expected, given this pattern of Snail expression, the levels of E-cadherin mRNA are low in metastatic AT2.1-GLI and AT6.3 cells, consistent with greater mesenchymal character, and are highest in the non-metastatic AT2.1 cells and in cyclopamine-treated AT6.3 cells (FIG. 6e).

Although human prostate cancer xenografts metastasize poorly in rodent hosts, the Hh-dependent induction of the same metastatic program noted in the rat Dunning model was achieved. Thus, 22RV1 cells displayed cyclopamine-sensitive invasive behavior in modified Boyden chamber assays, and cyclopamine sensitivity was bypassed by GLI overexpression (FIG. 6c). Invasion of the collagen matrix was also blocked by treatment with the Hh-neutralizing antibody, 5E1 (FIG. 6c), confirming a role for pathway activity and further implicating Hh ligand stimnulation in conferral of invasive behavior. At the level of gene expression, the three human prostate cancer cell lines examined all constitutively expressed high levels of Snail mRNA and very little E-cadherin mRNA (FIG. 6d,e). Treatment with cyclopamine confirmed that Snail expression is driven by Hh pathway activity and that increased expression of E-cadherin is associated with reduced Snail expression in these human cells (FIG. 6d,e). A third gene, Ndrg1, has been specifically associated with suppression of the metastatic phenotype, although without appreciable affects on proliferation, in prostate and colon cancer (Guan (2000), supra; Bandyopadhyay, S. et al. The Drg-1 gene suppresses tumor metastasis in prostate cancer. Cancer Res. 63, 1731-6 (2003)). This gene, like E-cadherin, is expressed in benign and non-metastatic tumor cells (Bandyopadhyay (2003), supra) but is not expressed in metastatic tumor cells unless Hi pathway blockade is imposed with cyclopamine (FIG. 6f).

Human prostate tumors are usually indolent, but approximately one in eight manifests the ability to metastasize and ultimately cause death. As metastatic potential is the critical determinant of clinical outcome, prognostic and therapeutic improvements in the management of prostate cancer require an understanding of metastatic potential and its underlying mechanisms. The results here indicate that Hh pathway activity promotes the ability of prostate cancer cells to proliferate indefinitely, but also implements a metastatic program that renders these tumors rapidly lethal.

As shown herein, cyclopamine suppression of Hh pathway activity results in a complete regression of human prostate cancer xenografts, and this regression persists indefinitely (currently up to 170 days) following cessation of treatment. This requirement for Hh pathway activity in tumor survival and growth suggests the existence of a Hh-dependent tumor stem cell, and raises a question as to the origin of this cell. It was also shown that Hh signaling activity is required in regeneration of prostate epithelium ablated by androgen deprivation, thus implicating pathway activity in maintenance or expansion of epithelial progenitors. The simplest interpretation of these results is that tumor stem cells in prostate cancer may arise from prostate epithelial stem cells or progenitors, with a similar role for pathway activity in expansion and maintenance of these tissue stem cells. Consistent with such a role for pathway activity, normal human prostate epithelial cells can be immortalized by overexpression of GLI, and these immortalized cells grow readily as tumors when inoculated subcutaneously in nude mice (data not shown). All of these findings are consistent with recent studies suggesting that a small fraction of the cells within solid tumors may be responsible for tumor growth and that these tumor stem cells share certain characteristics of stem or progenitor cells within the tissue of tumor origin (Al-Hajj, M., Wicha, M. S., Benito-Hernandez, A., Morrison, S. J. & Clarke, M. F. Prospective identification of tumorigenic breast cancer cells. Proc. Natl. Acad. Sci. USA 100, 3983-8 (2003); Singh, S. K. et al. Identification of a cancer stem cell in human brain tumors. Cancer Res. 63, 5821-8 (2003); and Kondo, T., Setoguchi, T. & Taga, T. Persistence of a small subpopulation of cancer stem-like cells in the C6 glioma cell line. Proc. Natl. Acad. Sci. USA 101, 781-6 (2004)). The requirement for Hh pathway activity in regeneration of prostate epithelium together with the previously demonstrated expression of pathway components in the context of airway epithelial injury (Watkins, D. N. et al. Hedgehog signalling within airway epithelial progenitors and in small-cell lung cancer. Nature 422, 313-7 (2003)) suggests the role and potential therapeutic utility of pathway activation in repair of diseased or injured endodermal organs.

In addition to its role in primary growth of tumor cells, the data presented herein support a distinct pathway role in activating a program of gene expression and cell behavior that fosters tumor metastasis. This program promotes mesenchymal as opposed to epithelial character, and includes suppression of Ndrgl, a gene whose expression is known to block metastasis. Pathway activity also dramatically increases invasiveness in modified Boyden chamber assays, widely considered as a correlate of the metastatic phenotype. Thus, although the more rapid rate of growth produced by pathway activation may contribute to metastasis (Chambers, A. F., Groom, A. C. & MacDonald, L. C. Dissemination and growth of cancer cells in metastatic sites. Nat. Rev. Cancer 2, 563-72 (2002)), the changes in gene expression and the increase in cell invasiveness that is noted here constitute a distinct metastatic program that is also activated by Hh pathway stimulation. These dual roles of Hh pathway activity in promoting growth and metastasis suggest that assessment and manipulation of Hh pathway activity may provide an important clinical avenue for the diagnosis and treatment of advanced prostate cancer.

TABLE 4

Oligonucleotide primers for quantitative real-time (*) and
conventional (#) amplification of reverse transcribed
mRNA (RT-PCR)

| | Forward | Reverse |
|---|---|---|
| Gene | SEQ ID NO'S 1 to 17 | SEQ ID NO'S 18 to 34 |
| PATCHED * | CGATGGAGTCCTTGCCTACAA | CCACCAGACGCTGTTTAGTCA |
| PATCHED # | CGCCTATGCCTGTCTAACCATGC | TAAATCCATGCTGAGAATTGCA |
| GLI # | TACTCACGCCTCGAAAACCT | GTCTGCTTTCCTCCCTGATG |
| SHH # | CAGCGACTTCCTCACTTTCC | GGAGCGGTTAGGGCTACTCT |
| IHH # | CCCCCTCCACTCCAATAAAT | AAAATTCTCCCATGGGCTTC |
| NESTIN * | CCAGGAGCCACTGAAGACTC | CCTTTCCCAGGTTCTCTTCC |
| PHOSPHOGLY- | CAGTTTGGAGCTCCTGGAAG | TGCAAATCCAGGGTGCAGTG |
| CERATE KINASE *# | | |
| Smoothened | TTACCTTCAGCTGCCACTTCTACG | GCCTTGGCAATCATCTTGCTCT |
| | | TC |
| c-Myc * | GGTGGAAAACCAGGTAAGCA | CCTTCTCCTCTGCCATCTTG |
| CyclinD * | GAGGAAGAGGAGGAGGAGGA | GAGATGGAAGGGGGAAAGAG |
| SNAIL | GGTTCTTCTGCGCTACTGCT | TAGGGCTGCTGGAAGGTAAA |
| Rat Snair * | CCGCCGGAAGCCCAACTAT | CCAGGAGAGAGTCCCAGATG |
| E-Cadherin * | CGACCCAACCCAAGAATCTA | AGGCTGTGCCTTCCTACAGA |
| Rat Cadherin 1 * | GAAGGCCTAAGCACAACAGC | ACGGTGTACACAGCATTCCA |
| Ndrg1 * | AATACCGAGTTAGGCGCAGTATGG | AATACCGAGTTAGGCGCAGTATGG |
| Rat Patched * | TAATCTCGAGACCAACGTGGAGGA | TGGTCAGGACATTAGCGCCTTCTT |
| Mouse Patched * | ATGCTCCTTTCCTCCTGAAACC | TGAACTGGGCAGCTATGAAGTC |

Although the invention has been described with reference to the above examples, it will be understood that modifications and variations are encompassed within the spirit and scope of the invention. Accordingly, the invention is limited only by the following claims.

What is claimed is:

1. A method of reducing or inhibiting proliferation or metastasis of cells of a prostate tumor characterized by elevated Hedgehog (Hh) pathway activity as compared with a normal cell, comprising contacting the cells with at least one Hh pathway antagonist, thereby reducing or inhibiting proliferation of the cells of the prostate tumor.

2. The method of claim 1, wherein the prostate tumor is a malignant tumor.

3. The method of claim 1, wherein the elevated Hh pathway activity comprises elevated ligand stimulated Hh pathway activity.

4. The method of claim 3, wherein the ligand comprises Sonic hedgehog (SHH) or Indian hedgehog (IHH) or a combination thereof.

5. The method of claim 1, wherein the Hh pathway antagonist comprises a steroidal alkaloid or derivative thereof.

6. The method of claim 5, wherein the steroidal alkaloid is cyclopamine.

7. The method of claim 1, wherein the Hh pathway antagonist is a nucleic acid or a protein molecule.

8. The method of claim 7, wherein the protein molecule is an antibody.

9. The method of claim 1, further comprising contacting the cells with a chemotherapeutic agent.

10. The method of claim 6, further comprising contacting the cells with an antibody.

11. The method fo claim 10, wherein the antibody is anti-Hh antibody.

12. A method of ameliorating a prostate tumor in a subject, comprising administering to the subject an Hh pathway antagonist, whereby the Hh pathway antagonist contacts cells of the tumor in the subject, thereby ameliorating the prostate tumor in the subject.

13. The method of claim 12, wherein the prostate tumor is a malignant tumor.

14. The method of claim 12, wherein the elevated Hh pathway activity comprises elevated ligand stimulated Hh pathway activity.

15. The method of claim 14, wherein the ligand comprises Sonic hedgehog (SHH) or Indian hedgehog (IHH).

16. The method of claim 12, wherein the Hh pathway antagonist comprises a steroidal alkaloid or derivative thereof.

17. The method of claim 16, wherein the steroidal alkaloid is cyclopamine.

18. The method of claim 12, further comprising administering to the subject a chemotherapeutic agent.

19. The method of claim 17, further comprising contacting the cells with an antibody.

20. The method fo claim 19, wherein the antibody is anti-Hh antibody.

21. The method of claim 12, wherein the Hh pathway antagonist is administered orally.

22. A method of identifying a prostate tumor of a subject amenable to treatment with a Hedgehog (Hh) pathway antagonist, comprising detecting elevated Hh pathway activity in a sample of cells from the subject as compared to Hh pathway activity in corresponding normal cells, thereby identifying a prostate tumor of a subject amenable to treatment with an Hh pathway antagonist.

23. The method of claim 22, wherein the cells are from a biopsy sample obtained from the subject.

24. The method of claim 22, wherein the cells are from a bodily fluid obtained from the subject.

25. The method of claim 22, wherein the elevated -Hh pathway activity comprises ligand stimulated Hh pathway activity.

26. The method of claim 22, comprising detecting elevated expression of at least one Hh pathway polypeptide.

27. The method of claim 26, wherein the Hh pathway polypeptide comprises an Bh ligand, an Hh ligand receptor, or a transcription factor.

28. The method of claim 27, wherein the Hh ligand comprises Sonic hedgehog (SHH), Indian hedgehog (IHH), or a combination thereof.

29. The method of claim 27, wherein the Hh ligand receptor comprises Patched.

30. The method of claim 27, wherein the transcription factor comprises a GLI-1 transcription factor.

31. The method of claim 26, which comprises detecting elevated levels of a polynucleotide encoding the Hh pathway polypeptide.

32. The method of claim 31, wherein the polynucleotide comprises RNA.

33. The method of claim 26, which comprises performing a reverse transcription-polymerase chain reaction.

34. The method of claim 26, which comprises detecting elevated levels of the Hh pathway polypeptide.

35. The method of claim 34, which comprises performing an immunoassay.

36. The method of claim 34, wherein the Hh pathway polypeptide comprises a transcription factor.

37. The method of claim 36, which comprises detecting increased binding activity of the transcription factor to a cognate transcription factor regulatory element.

38. The method of claim 36, which comprises detecting increased expression of a reporter gene comprising a cognate transcription factor regulatory element.

39. The method of claim 22, which comprises detecting altered expression of a transcriptional target of the Hh pathway.

40. The method of claim 39, which comprises detecting increased expression of a gene that is positively regulated by GLI-1 or GLI-2.

41. The method of claim 39, which comprises detecting decreased expression of gene that is negatively regulated by GLI-3.

42. The method of claim 22, comprising detecting decreased expression of at least one Hh pathway polypeptide.

43. The method of claim 42, wherein the Hh pathway polypeptide comprises a GLI-3 transcription factor.

44. The method of claim, 22, further comprising contacting cells of the sample with at least one Hh pathway antagonist, and detecting a decrease in Hh pathway activity in the cells following said contact, thereby confirming that the prostate tumor is amenable to treatment with an Hh pathway antagonist.

45. The method of claim 44, wherein the antagonist is cyclopamine.

46. The method of claim 45, further comprising contacting the cells with a chemotherapeutic agent.

47. The method of claim 45, further comprising contacting the cells with an anti-Hh antibody.

48. A method of identifying an agent useful for treating a prostate tumor or metastatsis wherein the tumor cells have elevated Hedgehog (Hh) pathway activity, comprising contacting a sample of cells of a prostate tumor with at least one test agent, wherein a decrease in Hh pathway activity in the presence of the test agent as compared to Hh pathway activity in the absence of the test agent identifies the agent as useful for treating the prostate tumor.

49. The method of claim 48, wherein the elevated Hh pathway activity comprises elevated ligand stimulated Hh pathway activity.

50. The method of claim 39, wherein the agent comprises an Hh pathway antagonist.

51. The method of claim 50, wherein the antagonist comprises steroidal alkaloid or a derivative thereof.

52. The method of claim 51, wherein the steroidal alkaloid is cyclopamine.

53. The method of claim 48, which is performed in a high throughput format.

54. The method of claim 53, comprising contacting samples of cells of a plurality of samples with at least one test agent.

55. The method of claim 54, wherein plurality of samples are obtained from a sinlgle-suect.

56. The method of claim 54, wherein the plurality of samples are obtained from different subjects.
